(19) United States
(12) Patent Application Publication
(10) Pub. No.: US 2012/0108503 A1
(43) Pub. Date: May 3, 2012

(54) METHODS FOR TREATING TYPE I DIABETES WITH LEPTIN AND LEPTIN AGONISTS

(75) Inventor: Roger H. Unger, Dallas, TX (US)

(73) Assignee: The board of Regents of the University of Texas System

(21) Appl. No.: 13/059,926

(22) PCT Filed: Aug. 20, 2009
(86) PCT No.: PCT/US09/54506

§ 371 Date: May 6, 2011

Related U.S. Application Data
(60) Provisional application No. 61/090,598, filed on Aug. 20, 2008.
(60) Provisional application No. 61/091,213, filed on Aug. 22, 2008.
(60) Provisional application No. 61/181,151, filed on May 26, 2009.

(51) Int. Cl. A61P 3/04; A61P 3/10; A61K 38/00; A61K 31/7088; A61K 38/22; A61K 38/28

(52) U.S. Cl. 514/4.8; 514/5.8; 514/6.5; 514/7.3; 514/44 R

(57) ABSTRACT

The present invention provides for methods of treating type I diabetes by inducing hyperleptinemia in subjects afflicted with type I diabetes. These methods can achieve normoglycemia and suppress hypergluconemia, and alleviate conditions associated with such, even in the absence of or at extremely low levels of adjunct insulin therapy, and without any appreciable increase in insulinogenesis.

This application claims benefit of U.S. Provisional Application Ser. No. 61/181,151, filed May 26, 2009, U.S. Provisional Application Ser. No. 61/091,213, filed Aug. 22, 2008, and U.S. Provisional Application Ser. No. 61/090,598, filed Aug. 20, 2008, the entire contents of each application being are hereby incorporated by reference.

This invention was made with government support under grant no. #5-R01-DK002700 awarded by National Institute of Diabetes and Digestive and Kidney Diseases, and the Veterans' Administration. The government has certain rights in the invention.

### BACKGROUND OF THE INVENTION

1. Field of the Invention

The present invention relates generally to the field of human cell biology and pathology, and more particularly to diabetes. Specifically, the invention provides methods for treating symptoms of type I diabetes and/or re-establishing normoglycemia in a type I diabetic subject by inducing hyperleptinemia in the subject.

2. Description of Related Art

Until the discovery of insulin in 1921 by Banting and Best (1990) type I diabetes (T1D) was a uniformly fatal illness. Ever since the introduction of insulin treatment, it has been assumed that only insulin can reverse this lethal catabolic syndrome, in which virtually all insulin-producing cells are destroyed by an autoimmune process.

The discovery in 1994 of the adipocyte hormone leptin (Zhang et al., 1994) identified a novel agent with a myriad of physiologic and pharmacologic effects. Leptin is a 16 kDa protein hormone that plays a key role in regulating energy intake and energy expenditure, including appetite and metabolism. Leptin is produced by adipose tissue and interacts with six types of receptor (LepRa-LepRf). LepRb is the only receptor isoform that contains active intracellular signaling domains.

Among the effects caused by leptin are a robust blood glucose-lowering effect observed in normal rodents (Koyama et al., 1997) and in rodents with partial insulin deficiency induced by streptozotocin (STZ) (Chinookoswong et al., 1999; Miyanaga et al., 2003). It is used therapeutically in patients with lipodystrophic diabetes (Oral et al., 2002). However, because none of these leptin-responsive diabetic models were completely insulin-deficient, these findings have been interpreted by the art to indicate that the antihyperglycemic effects of leptin treatment result from an increase in insulin sensitivity and potentiation of residual levels of endogenous insulin (see, e.g., Miyanaga et al., 2003). The idea that leptin had actually replaced the therapeutic actions of insulin and/or may be administered as an anti-diabetic agent, anti-hyperglycemic agent, and/or an anti-hypergluconemia agent, independent of or in the absence of clinically relevant insulin activity, has never been appropriately tested or suggested.

### SUMMARY OF THE INVENTION

Thus, in accordance with the present invention, there is provided a method of treating type I diabetes comprising providing to a subject diagnosed with type I diabetes: a therapeutically effective amount of (a) a leptin, a leptin agonist, or a leptin derivative; and (b) no more than about 10% of a normal daily dosage of insulin supplementation. In certain embodiments, between 5% and 10%, inclusive; less than 5%; or between zero and 5%, including 4%, 3%, 2%, 1%, 0.5% and 0.1%; of a normal daily dose on insulin supplementation is employed in accordance with said method. The provision of said therapeutically effective amount of a leptin, a leptin agonist, or a leptin derivative may comprise the provision of a leptin polypeptide, leptin agonist poypeptide, or leptin derivative polypeptide; alternatively, the provision of said therapeutically effective amount may comprise the provision of a leptin-, a leptin agonist-, or a leptin derivative-encoding nucleic acid, expression construct, or vector, as disclosed below.

In another embodiment, there is provided a method of restoring normoglycemia in a subject diagnosed with or otherwise having type I diabetes comprising: inducing hyperleptinemia in said subject, wherein said inducing comprises the provision of a therapeutically effective amount of a leptin, a leptin agonist, or a leptin derivative; wherein said subject receives no more than about 10% of a normal daily dosage of insulin supplementation. In certain embodiments, between 5% and 10%, inclusive; less than 5%; or between zero and 5% including 4%, 3%, 2%, 1%, 0.5% and 0.1%; of a normal daily dose on insulin supplementation is employed in accordance with said method. The provision of said therapeutically effective amount of a leptin, a leptin agonist, or a leptin derivative may comprise the provision of a leptin polypeptide, leptin agonist poypeptide, or leptin derivative polypeptide; alternatively, the provision of said therapeutically effective amount may comprise the provision of a leptin-, a leptin agonist-, or a leptin derivative-encoding nucleic acid, expression construct, or vector, as disclosed below.

Still another embodiment comprises a method of reducing, suppressing, attenuating, or inhibiting hyperglucogonemia or a condition associated with hyperglucogonemia in a subject diagnosed with type I diabetes comprising: inducing hyperleptinemia in said subject, wherein said inducing comprises the provision of a therapeutically effective amount of a leptin, a leptin agonist, or a leptin derivative; wherein said subject receives no more than about 10% of a normal daily dosage of insulin supplementation. In certain embodiments, between 5% and 10%, inclusive; less than 5%; or between zero and 5% including 4%, 3%, 2%, 1%, 0.5% and 0.1%; of a normal daily dose on insulin supplementation is employed in accordance with said method. The provision of said therapeutically effective amount of a leptin, a leptin agonist, or a leptin derivative may comprise the provision of a leptin polypeptide, leptin agonist poypeptide, or leptin derivative polypeptide; alternatively, the provision of said therapeutically effective amount may comprise the provision of a leptin-, a leptin agonist-, or a leptin derivative-encoding nucleic acid, expression construct, or vector, as disclosed below.

Yet a further embodiment comprises a method of reducing HbAlc in a subject diagnosed with type I diabetes comprising inducing hyperleptinemia in said subject, wherein said inducing comprises the provision of a therapeutically effective amount of a leptin, a leptin agonist, or a leptin derivative; wherein said subject receives no more than about 10% of a normal daily dosage of insulin supplementation. In certain embodiments, between 5% and 10%, inclusive; less than 5%; or between zero and 5% including 4%, 3%, 2%, 1%, 0.5% and 0.1%; of a normal daily dose on insulin supplementation is employed in accordance with said method. The provision of said therapeutically effective amount of a leptin, a leptin agonist, or a leptin derivative may comprise the provision of a leptin polypeptide, leptin agonist poypeptide, or leptin derivative polypeptide; alternatively, the provision of said therapeutically effective amount may comprise the provision of a leptin-, a leptin agonist-, or a leptin derivative-encoding nucleic acid, expression construct, or vector, as disclosed below.

The hyperleptinemia that is induced by way of the methods described above and below may be characterized by plasma leptin levels of greater than 10 ng/ml, 20 ng/ml, 50 ng/ml, 100 ng/ml, 200 ng/ml, 300 ng/ml or 400 ng/ml. The method may result in a venous or capillary fasting blood glucose (FBG) levels of less than 200 mg/dl, less than 175 mg/dl, less than 150 mg/dl, less than 140 mg/dl, less than 130 mg/dl, less than 126 mg/dl, less than 120 mg/dl, or less than 115 mg/dl, less than 110 mg/dl, or less than 100 mg/dl.

The subject may be a non-human animal, such as a mouse or rat, or a human. The subject may suffer from autoimmune type I diabetes, or from chemically-induced type I diabetes. Inducing may comprise administering a leptin, a leptin agonist, or a leptin derivative to said subject. The leptin agonist may comprise metreleptin (SEQ ID NO: 13). Inducing may also comprise administering an expression cassette comprising a promoter operably linked to a leptin-encoding nucleic acid or a leptin agonist-encoding nucleic acid to said subject. The promoter may be a tissue specific or constitutive promoter. The expression cassette may be comprised within a lipid vehicle and/or comprised within a replicable expression construct. The replicable expression construct may be a non-viral construct or a viral construct, such as an adenoviral construct, an adeno-associated viral construct, a pox-viral construct, a retroviral construct, or a herpesviral construct.

The one or more symptoms of untreated or uncontrolled type I diabetes may comprise excess gluconeogenesis, excess glycogenolysis, hyperglycemia, hyperglucagonemia, ketosis, diabetic ketoacidosis, hypertriglyceridemia, elevated plasma free fatty acid, weight loss, catabolic syndrome, terminal illness, hypertension, diabetic nephropathy, renal insufficiency, renal failure, hyperphagia, muscle wasting, diabetic neuropathy, diabetic retinopathy, or diabetic coma.

As mentioned above and below, the insulin daily dosage that may be provided in accordance with the disclosed and claimed methods may between 10-15% of the normal daily dosage, 5-10% of the normal daily dosage, or less than 5% of the normal daily dosage, or between zero and 5% of the normal daily dosage, including no insulin. The insulin dosage may be reduced following initiation of leptin or leptin agonist provision. In certain embodiments, the subject may be essentially devoid of detectable endogenous insulin in blood, plasma, or serum, or is essentially devoid of insulin activity. “Essentially devoid of insulin activity” means that any insulin that may be detected in subjects constitutes a physiologically or clinically non-relevant amount.

A “physiologically non-relevant amount” or alternatively, a “clinically non-relevant amount” means an amount of an agent, such as an insulin, whether endogenous insulin or exogenous insulin” that does is not sufficient to attenuate, inhibit, suppress, reduce or ameliorate a type I diabetic phenotype. Thus, subjects that possess or display “physiologically non-relevant amount,” or alternatively, a “clinically non-relevant amount,” and are thus, “essentially devoid of insulin” or “essentially devoid of insulin activity” are distinguished from non-diabetic subjects and are also distinguished from subjects that possess or display clinical manifestations of type II diabetic phenotype, which type II phenotype is predominantly characterized by, for example, insulin resistance and insulin insensitivity. In such type II diabetic subjects, or such subjects possessing manifestations of a type II diabetic phenotype, subjects are predominantly resistant and/or insensitive to any amount of endogenous insulin or insulin activity that is present in such subjects. Thus, such endogenous insulin may be potentiated by the provision of insulin sensitizing agents. In contrast, a type I diabetic subject is diagnosed on the basis of insulin dependence, not insulin insensivity.

Accordingly, in certain embodiments, the subject may be essentially devoid of endogenous insulin or endigenour insulin activity, which comprises a lack of detectable insulin production or expression. Such a lack of dectable insulin production or expression may be determined as determined by, for example, measurement of C— peptide levels, preproinsulin mRNA or polypeptide levels, proinsulin polypeptide levels, or mature insulin levels, as is known in the art.

The method may comprise systemically administering to said subject leptin, a leptin agonist, or a expression cassette encoding the same. Systemically administering may comprise intravenous, intra-muscular, subcutaneous, intraperitoneal, transdermal or intra-arterial administration. The method may also comprise administering directly to a tissue of said subject leptin, a leptin agonist, or a expression cassette encoding the same, for example, muscle or liver. The method may comprise multiple administrations, such as 2, 3, 4, 5, 6, 7, 8, 9, 10, 15, 20, 25, 30, 40, 50, 75, 100, 150, 200, 300, 400, 500 or more administrations. The multiple administrations may be separated by 12 hours, 1 day, 2 days, 3, days, 4, days, 5 days, 6 days, 1 week, 2 weeks, one month, two months, three months, 6 months or more.

As used herein the specification, “a” or “an” may mean one or more. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D. Hyperleptinemia reverses abnormalities of uncontrolled autoimmune diabetes in the absence of insulin. Comparison of mean (±SEM) (FIG. 1A) leptin levels, (FIG. 1B) blood glucose levels, (FIG. 1C) body weight and food intake (shaded dark area=Adv-leptin), and (FIG. 1D) plasma glucagon levels in diabetic NOD mice after either treatment with Adv-leptin (▪) (N=9) or injection of Adv-β-gal (□) as a control (N=6). Plasma glucagon levels of diabetic NOD mice were obtained 30 days after treatment with Adv-leptin (▪) (N=9) or injection of Adv-β-gal (□) (N=6). Glucagon levels of prediabetic NOD mice (N=6) are also displayed (▪). *p<0.01.

FIGS. 2A-D. Hyperleptinemia reverses abnormalities of uncontrolled chemical diabetes in the absence of insulin. Comparisons of mean (±SEM) (FIG. 2A) leptin levels, (FIG. 2B) plasma glucose levels and (FIG. 2C) body weight in untreated streptozotocin (STZ)-diabetic rats on an unrestricted diet (∘) (N=5), or, untreated streptozotocin (STZ)-diabetic rats pairfed to leptinized rats (Δ) (N=3) and in streptozotocin (STZ)-diabetic rats treated with Adv-leptin (▪) (N=6). (FIG. 2D) Blood glucose levels in untreated alloxan-diabetic rats (∘) (N=5) or treated with Adv-leptin (▪) (N=6).

FIGS. 3A-C. Hyperleptinemia reverses abnormalities of uncontrolled diabetes induced by a double dose of STZ. Comparison of mean (±SEM) (FIG. 3A) blood glucose levels treated with Adv-leptin (▪) (N=5) or untreated double dose STZ-diabetic rats (∘) (N=5). (FIG. 3B) Morphometric comparison of insulin-positive cells (mean±SEM) in pancreata of 4 untreated, 5 Adv-leptin treated STZ-diabetic rats and 3 normal non-diabetic controls. (FIG. 3C) Plasma glucagon levels 30 days after treatment (*p<0.01).

FIGS. 4A-C. Hyperleptinemia activates liver STAT-3 and down-regulates proteins of gluconeogenesis, while limiting postprandial hyperglycemia. Comparisons (mean±SEM) of relevant signal transcription factors for leptin and glucagon and their gluconeogenic targets in livers of untreated (□) (N=4) double-dose STZ-diabetic rats 3 days after Adv-leptin treatment (▪) (N=5) and 3 hours after insulin treatment (▪) (N=3). (FIG. 4A) Immunoblotting for P-STAT-3 and total STAT-3 (upper), and immunoblotting for P-CREB and total CREB (lower). Results are in densitometric units. (FIG. 4B) mRNA of phosphoenol pyruvate carboxykinase (PEPCK) and peroxisome proliferator activated receptor coactivator-1 (PGC-1a). (FIG. 4C) Postprandial rise above fasting levels in blood glucose of untreated STZ rats (□), Adv-leptin-treated STZ rats (▪) and nondiabetic rats (▪).

FIGS. 5A-D. Hyperleptinemia increases plasma IGF-1 and IGF-1 action on skeletal muscle, while restoring linear growth in severely insulin-deficient rats. Comparisons of (FIG. 5A) plasma IGF-1 and (FIG. 5B) liver IGF-1 mRNA 30 days after treatment and (FIG. 5C) phosphorylated IGF-1 receptor (P-IGF-1R) in skeletal muscle 3 days after treatment (densitometric units) in untreated (□) (N=4) and Adv-leptin-treated (▪) (N=4) double-dose-STZ-diabetic rats. (FIG. 5D) Appearance of a nondiabetic normal lean wild-type Zucker Diabetic Fatty (+/+) rat, a double-dose-STZ-diabetic littermate treated with Adv-leptin, and an untreated diabetic littermate. Note that, while both the leptinized and the untreated diabetic rats are slimmer than the nondiabetic wild-type control, the length of the leptinized rat is almost normal. Thus, the growth inhibition caused by insulin deficiency was corrected without insulin replacement.

FIG. 6. A long-term study showing a gradual return of hyperglycemia that nevertheless remains below pretreatment levels. The animals retained body weight and appeared to be in normal health. (▪=Adv-leptin; Δ=Adv-β-gal; * p<0.01; ** p<0.05).

FIG. 7. Hyperleptinemia increases activation of certain components of the insulin signaling pathway in skeletal muscle. Immunoblotting for phophoproteins of the insulin signaling transduction pathway in skeletal muscle of double-dose STZ-diabetic rats. Rats were untreated (□) (N=4), or they received Adv-leptin 3 days earlier (▪) (N 5) or insulin 3 hours earlier (□) (N=3). Results are expressed as densitometric units. Hyperglucagonemia is the cause of lethal components of insulin deficiency. Sustained glucagon suppression is the main benefit of hyperleptinemia.

FIGS. 8A-D. Plasma levels of type I diabetic NOD mice treated with subcutaneously infused leptin with insulin delivered from a subcutaneous pellet or with untreated controls infused with PBS (phosphate-buffered saline) and either fed ad lib pairfed to the leptin-treated group. (FIG. 8A) Plasma leptin levels. (FIG. 8B) Blood glucose levels. The broken line marks the normal fasting glucose level. (FIG. 8C) Hemoglobin Alc. The broken line marks the normal level. (FIG. 8D) Plasma free fatty acid (FFA) levels. The broken line marks the mean level in normal mice.

FIGS. 9A-D. Triacylglycerol (TG) levels and expression of transcription factors and enzymes involved in lipogenesis and cholesterologenesis in livers of type I diabetic NOD mice treated either with subcutaneously infused leptin, insulin delivered from a subcutaneous pellet compared to untreated controls infused with PBS and fed ad lib, or pairfed to the leptin-treated group. (FIG. 9A) Plasma triacylglycerol (TG) concentration. (FIG. 9B) Liver TG content. (FIG. 9C) Hepatic expression of transcription factors and enzymes involved in lipogenesis. (FIG. 9D) Hepatic expression of transcription factors and enzymes involved in cholesterologenesis.

FIGS. 10A-D. Plasma glucagon levels and activation of its transcription factor and target enzyme in livers of type I diabetic NOD mice treated either with subcutaneously infused leptin, insulin delivered from a subcutaneous pellet, and untreated controls infused with PBS and fed ad lib, or pairfed to the leptin-treated group. (FIG. 10A) Ratio of phosphorylated to total AMP-activated protein kinase (AMPK). (FIG. 10B) Plasma glucagon. (FIG. 10C) Ratio of phosphorylated to total cAMP-response element binding protein (CREB). (FIG. 10D) mRNA of phosphoenolpyruvate carboxykinase.

FIG. 11. Comparison of plasma glucose levels in type I diabetic NOD mice treated either with twice daily injection of leptin at the indicated total doses plus insulin at a total dose of 0.02 U/d or 0.02 U/d only, or insulin at a dose of 0.2 U/d, considered optimal.

FIG. 12. Flow diagram for leptin clinical study. The patients with T1DM are expected to participate for 3 months. The arrows indicate the times at which various tests will be conducted. Shaded regions indicate in-patient evaluation.

### DESCRIPTION OF ILLUSTRATIVE EMBODIMENTS

Hyperleptinemia reduces hyperglycemia in streptozotocin diabetic rats with partial insulin deficiency (Hidaka et al., 2002; Lin et al., 2002), and can directly activate certain components of the insulin signal transduction pathway in normal rats (Kim et al., 2004). However, the possibility that leptin or a leptin agonist could rescue insulin-deficient animals from ketoacidosis and death had never been tested. Here, the inventor shows that hyperleptinemia, for example sustained, supraphysiologic hyperleptinemia, induced by a single injection of Adv-leptin or via leptin infusion, reverses all of the measurable consequences of insulin deficiency, whether autoimmune or chemically-induced, and restores health in rodents without preproinsulin mRNA in pancreas or in ectopic sites.

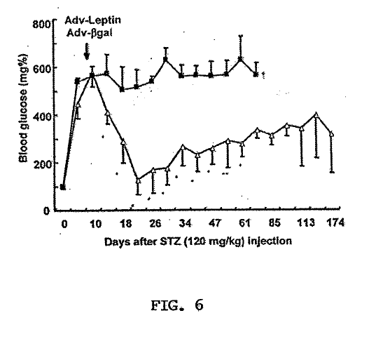

The single most striking feature of this study was the dramatic clinical improvement achieved without insulin in terminally ill rodents. Without exception, every animal exhibited significant daily improvement, becoming normoglycemic and in a normal state of health within 5-12 days. The results were similar in mice with autoimmune destruction and in rats with chemical destruction of β-cells, although the timing differed. In all models, weight loss was arrested and weight gain resumed despite the virtual absence of body fat. Although the anti-hyperglycemic effect of hyperleptinemia in STZ rats wanes 2-3 weeks thereafter, the hyperglycemia remains well below the extremely elevated pretreatment levels—even at 25 weeks after Adv-leptin treatment (FIG. 6), perhaps because the adenovirally-induced hyperleptinemia persists at low levels for many weeks (Higa et al., 1999), and glucagon remains suppressed.

Earlier work from the inventor's lab had demonstrated that hyperglucagonemia is present in insulin deficiency states (Muller et al., 1971) and that its suppression by somatostatin prevents the hepatic overproduction of glucose and ketones in uncontrolled diabetes for several hours (Dobbs et al., 1975; Gerich et al., 1975; Unger and Orci, 1975). In this study, the inventor found that sustained hyperleptinemia can also profoundly suppress diabetic hyperglucagonemia to normal for extended periods of time. In addition, hyperleptinemia can inhibit glucagon's gluconeogenic action on the liver, as manifested by a reduction in P-CREB and PEPCK and PGC1α.

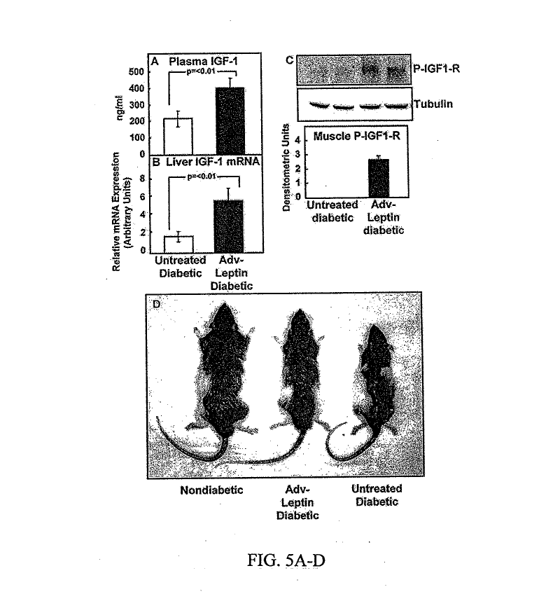

Although leptin stimulates MAPK phosphorylation almost 4-fold in the livers of normal rats (Kim et al., 2004; Szanto and Kahn, 2000), in the insulin-deprived animals studied here, hyperleptinemia had no such effect. There was, however, evidence of insulin-like effects in an extrahepatic target of insulin, the skeletal muscle, where P-STAT3 increased almost 10-fold. In confirmation of the report of Kim et al. (2004), there was an increase in skeletal muscle P-IRS-1, P-ERK and P13K to ˜65%, 44% and 30%, respectively, of the insulin-induced increase. These effects could have been mediated by IGF-1, which was upregulated in the liver of leptinized rats and which was increased in plasma. There was increased phosphorylation of IGF-1 receptor in skeletal muscle, which is consistent with this, as is the striking restoration of linear growth in the absence of any insulin (FIG. 5C).

Based on this evidence, the inventor speculates that the reversal by hyperleptinemia of the protein catabolism of total insulin deficiency was the result of suppression of hyperglucagonemia and its protein-catabolic hepatic actions on liver, combined with upregulation of IGF-1, a protein-anabolic hormone. Whatever their mechanisms, these results constitute the first report of successful treatment of total insulin deficiency without insulin and suggest that uncontrolled diabetes can be rescued without insulin by agents that eliminate glucagon-mediated hepatic overproduction of glucose and ketones and that improve glucose utilization in skeletal muscle. Consequently, it is hoped that these findings will lead to the development of glucagon-suppressing/blocking agents that might supplement or even replace insulin treatment in the management of Type I diabetes.

### I. TYPE I DIABETES

A. General Background

Type I diabetes (T1D), or diabetes mellitus type I, is a form of diabetes mellitus. Type I diabetes is an autoimmune disease that results in the permanent destruction of insulin-producing β cells of the pancreas. Type I is lethal unless treatment with exogenous insulin via injections replaces the missing hormone, or a functional replacement for the destroyed pancreatic beta cells is provided (such as via a pancreas transplant).

Type I diabetes (formerly known as “childhood,” “juvenile” or “insulin-dependent” diabetes) is not exclusively a childhood problem. The adult incidence of type I is noteworthy—many adults who contract type I diabetes are misdiagnosed with type 2 due to the misconception of type I as a disease of children—and since there is no cure, all children with type I diabetes will grow up to be adults with type I diabetes.

There is currently no preventive measure that can be taken against type I diabetes. Most people affected by type I diabetes are otherwise healthy and of a healthy weight when onset occurs, but they can lose weight quickly and dangerously, if not diagnosed in a relatively short amount of time. Diet and exercise cannot reverse or prevent type I diabetes. Although there are clinical trials ongoing that aim to find methods of preventing or slowing its development, so far none have proven successful, at least on a permanent basis.

The most useful laboratory test to distinguish type I from type II diabetes is the C-peptide assay, which is a measure of endogenous insulin production since external insulin (to date) has included no C-peptide. However, C-peptide is not absent in type I diabetes until insulin production has fully ceased, which may take months. The presence of anti-islet antibodies (to Glutamic Acid Decarboxylase, Insulinoma Associated Peptide-2 or insulin), or lack of insulin resistance, determined by a glucose tolerance test, would also be suggestive of type I. As opposed to that, many type II diabetics still produce insulin internally, and all have some degree of insulin resistance. Testing for GAD 65 antibodies has been proposed as an improved test for differentiating between type I and type II diabetes.

The cause of type I diabetes is still not fully understood. Some theorize that type I diabetes could be a virally-induced autoimmune response. Autoimmunity is a condition where one's own immune system “attacks” structures in one's own body either destroying the tissue or decreasing its functionality. In the proposed scenario, pancreatic beta cells in the Islets of Langerhans are destroyed or damaged sufficiently to abolish endogenous insulin production. This etiology makes type I distinct from type II diabetes mellitus. It should also be noted that the use of insulin in a patient's diabetes treatment protocol does not render them as having type I diabetes, the type of diabetes a patient has is determined only by disease etiology. The autoimmune attack may be triggered by reaction to an infection, for example by one of the viruses of the Coxsackie virus family or German measles, although the evidence is inconclusive.

This vulnerability is not shared by everyone, for not everyone infected by these organisms develops type I diabetes. This has suggested a genetic vulnerability and there is indeed an observed inherited tendency to develop type I. It has been traced to particular HLA genotypes, though the connection between them and the triggering of an auto-immune reaction is poorly understood. Wide-scale genetic studies have shown links between genetic vulnerabilities for type I diabetes and Multiple Sclerosis and Crohn's Disease.

Some researchers believe that the autoimmune response is influenced by antibodies against cow's milk proteins. A large retrospective controlled study published in 2006 strongly suggests that infants who were never breastfed had a risk for developing type I diabetes twice that of infants who were breastfed for at least three months. The mechanism, if any, is not understood. No connection has been established between autoantibodies, antibodies to cow's milk proteins, and type I diabetes. A subtype of type I (identifiable by the presence of antibodies against β cells) typically develops slowly and so is often confused with type II. In addition, a small proportion of type I cases have the hereditary condition maturity onset diabetes of the young (MODY) which can also be confused with type II.

Vitamin D in doses of 2000 IU per day given during the first year of a child's life has been connected in one study in Northern Finland (where intrinsic production of Vitamin D is low due to low natural light levels) with an 80% reduction in the risk of getting type I diabetes later in life. Some suggest that deficiency of Vitamin D₃ (one of several related chemicals with Vitamin D activity) may be an important pathogenic factor in type I diabetes independent of geographical latitude.

Some chemicals and drugs specifically destroy pancreatic cells. Vacor (N-3-pyridylmethyl-N′-p-nitrophenyl urea), a rodenticide introduced in the United States in 1976, selectively destroys pancreatic β cells, resulting in type I diabetes after accidental or intentional ingestion. Vacor was withdrawn from the U.S. market in 1979. Zanosar® is the trade name for streptozotocin, an antibiotic and antineoplastic agent used in chemotherapy for pancreatic cancer, that kills β cells, resulting in loss of insulin production.

Other pancreatic problems, including trauma, pancreatitis or tumors (either malignant or benign), can also lead to loss of insulin production. The exact cause(s) of type I diabetes are not yet fully understood, and research on those mentioned, and others, continues.

B. Treatments

Untreated type I diabetes can lead to one form of diabetic coma, diabetic ketoacidosis, which can be fatal. Other aspects of the disease include excess gluconeogenesis, excess glycogenolysis, hyperglycemia, hyperglucagonemia, ketosis, hypertriglyceridemia, elevated plasma free fatty acid, weight loss, hypertension, diabetic nephropathy, renal insufficiency, renal failure, hyperphagia, muscle wasting, diabetic neuropathy, and diabetic retinopathy

Type I is treated with insulin replacement therapy—usually by injection or insulin pump, along with attention to dietary management, typically including carbohydrate tracking, and careful monitoring of blood glucose levels using glucose meters. At present, insulin treatment must be continued for a lifetime; this will change if better treatment, or a cure, is discovered. Continuous glucose monitors have been developed which can alert patients to the presence of dangerously high or low blood sugar levels, but the lack of widespread insurance coverage has limited the impact these devices have had on clinical practice so far.

There are both short- and long-term disadvantages to insulin therapy. The main short-term issue concern with insulin monotherapy is the instability of the daily glucose profiles achieved by peripheral injections of insulin. Even optimally controlled patients with at target HgbAlc values have daily spikes of hyperglycemia, with occasional hypoglycemic dips. This may be the result of the enormous anatomical disadvantage of peripherally injected insulin, which cannot meet the high insulin requirements of proximal targets such as alpha cells and hepatocytes without far exceeding the insulin requirements of distal targets such as muscle and fat. The intra-islet concentration of endogenous insulin that perfuses alpha cells in normal islets has been estimated to be over 20× higher than the levels generated by peripheral injection, and the concentration of endogenous insulin perfusing the liver is 4- to 5-times higher. This means that even a high concentration of exogenous insulin in peripheral plasma may not approach the physiologic levels of endogenous insulin that perfuse these two proximal insulin targets, which control endogenous glucose production.

A second important disadvantage of injected insulin is its inability to respond on a minute-to-minute basis to changes in need, in particular, to lower it instantly when glucose levels are falling. These facts suggest that, if the wild and inappropriate oscillations of insulin and glucagon that create inappropriate swings in hepatic glucose production could be chronically stabilized by suppressing glucagon independently of insulin, the total daily dose of insulin could be reduced to levels that would manage postprandial hyperglycemia and nothing else. The hope is that this would establish a more stable pattern of glucoregulation.

A third important contributing factor to glucose lability is lipolytic lability, which intermittently floods the target tissues of insulin with fatty acids that impair sensitivity to insulin action on glucose metabolism. This contributes to instability of glucose levels, which can fluctuate from dangerously low levels of hypoglycemia to undesirably high hyperglycemia, making frequent blood glucose determination and multiple insulin injections mandatory, thereby significantly lowering the quality of life for patients.

The major long-term effect of insulin therapy is insulin resistance, a well characterized component of type I diabetes. As in T2DM, the degree of insulin resistance is closely associated with risk of cardiovascular disease. The high prevalence of coronary artery disease among patients with T1DM is traditionally ascribed to the disease rather than to life-long insulin monotherapy. The role of insulin in the macrovascular complications of T1DM deserves to be examined more closely, given the relationship between the diet-driven endogenous hyperinsulinemia of obesity and the metabolic syndrome, particularly in insulin-resistant patients treated with U-500 insulin. Insulin is a powerful lipogenic force; a life-time of exogenous hyperinsulinemia in T1DM could also cause a form of metabolic syndrome, with insulin resistance, hyperlipidemia, hypercholesterolemia, coronary artery disease and lipotoxic cardiomyopathy and occasionally obesity.

In more extreme cases, a pancreas transplant can help restore proper glucose regulation. However, the surgery and accompanying immunosuppression required is considered by many physicians to be more dangerous than continued insulin replacement therapy and is therefore often used only as a last resort (such as when a kidney must also be transplanted or in cases where the patient's blood glucose levels are extremely volatile). Experimental replacement of β cells (by transplant or from stem cells) is being investigated in several research programs and may become clinically available in the future. Thus far, β cell replacement has only been performed on patients over age 18, and with tantalizing successes amidst nearly universal failure.

Pancreas transplants are generally recommended if a kidney transplant is also necessary. The reason for this is that introducing a new kidney requires taking immunosuppressive drugs anyway, and this allows the introduction of a new, functioning pancreas to a patient with diabetes without any additional immunosuppressive therapy. However, pancreas transplants alone can be wise in patients with extremely labile type I diabetes mellitus.

Less invasive than a pancreas transplant, islet cell transplantation is currently the most highly used approach in humans to temporarily cure type I diabetes. In one variant of this procedure, islet cells are injected into the patient's liver, where they take up residence and begin to produce insulin. The liver is expected to be the most reasonable choice because it is more accessible than the pancreas, and the islet cells seem to produce insulin well in that environment. The patient's body, however, will treat the new cells just as it would any other introduction of foreign tissue. The immune system will attack the cells as it would a bacterial infection or a skin graft. Thus, the patient also needs to undergo treatment involving immunosuppressants, which reduce immune system activity.

Recent studies have shown that islet cell transplants have progressed to the point that 58% of the patients in one study were insulin independent one year after the operation. Ideally, it would be best to use islet cells which will not provoke this immune reaction, but investigators are also looking into placing islets into a protective coating which enables insulin to flow out while protecting the islets from white blood cells.

### II. LEPTIN

Leptin (Greek leptos meaning thin) is a 16 kDa protein hormone that plays a key role in regulating energy intake and energy expenditure, including appetite and metabolism. Leptin is one of the most important adipose derived hormones.

The effects of leptin were observed by studying mutant obese mice that arose at random within a mouse colony at the Jackson Laboratory in 1950. These mice were massively obese and hyperphagic. Leptin itself was discovered in 1994 by Jeffrey M. Friedman and colleagues at the Rockefeller University through the study of such mice. The Ob(Lep) gene (Ob for obese, Lep for leptin) is located on chromosome 7 in humans. Leptin is produced by adipose tissue and interacts with six types of receptor (LepRa-LepRf). LepRb is the only receptor isoform that contains active intracellular signaling domains. This receptor is present in a number of hypothalamic nuclei. Leptin binds to the ventromedial nucleus of the hypothalamus, known as the “appetite center.” Leptin signals to the brain that the body has had enough to eat, or satiety. A very small group of humans possess homozygous mutations for the leptin gene which leads to a constant desire for food, resulting in severe obesity. This condition can be successfully treated by the administration of recombinant human leptin.

Thus, circulating leptin levels give the brain input regarding energy storage so it can regulate appetite and metabolism. Leptin works by inhibiting the activity of neurons that contain neuropeptide Y (NPY) and agouti-related peptide (AgRP), and by increasing the activity of neurons expressing α-melanocyte-stimulating hormone (α-MSH). The NPY neurons are a key element in the regulation of appetite; small doses of NPY injected into the brains of experimental animals stimulates feeding, while selective destruction of the NPY neurons in mice causes them to become anorexic. Conversely, α-MSH is an important mediator of satiety, and differences in the gene for the receptor at which α-MSH acts in the brain are linked to obesity in humans.

There is some controversy regarding the regulation of leptin by melatonin during the night. One research group suggested that increased levels of melatonin caused a downregulation of leptin. However, in 2004, Brazilian researchers found that in the presence of insulin, “melatonin interacts with insulin and upregulates insulin-stimulated leptin expression,” therefore causing a decrease in appetite whilst sleeping.

It is unknown whether leptin can cross the blood-brain barrier to access receptor neurons, because the blood-brain barrier is somewhat absent in the area of the median eminence, close to where the NPY neurons of the arcuate nucleus are. It is generally thought that leptin might enter the brain at the choroid plexus, where there is intense expression of a form of leptin receptor molecule that could act as a transport mechanism.

Once leptin has bound to the Ob-Rb receptor, it activates the Stat3, which is phosphorylated and travels to the nucleus to, presumably, effect changes in gene expression. One of the main effects on gene expression is the down-regulation of the expression of endocannabinoids, responsible for increasing appetite. There are other intracellular pathways activated by leptin, but less is known about how they function in this system. In response to leptin, receptor neurons have been shown to remodel themselves, changing the number and types of synapses that fire onto them.

Although leptin is a circulating signal that reduces appetite, in general, obese people have an unusually high circulating concentration of leptin. These people are said to be resistant to the effects of leptin, in much the same way that people with type II diabetes are resistant to the effects of insulin. The high sustained concentrations of leptin from the enlarged adipose stores result in leptin desensitization. The pathway of leptin control in obese people might be flawed at some point so the body doesn't adequately receive the satiety feeling subsequently to eating.

In mice, leptin is also required for male and female fertility. In mammals such as humans puberty in females is linked to a critical level of body fat. When fat levels fall below this threshold (as in anorexia), the ovarian cycle stops and females stop menstruating. Leptin is also strongly linked with angiogenesis, increasing VEGF levels.

The body's fat cells, under normal conditions, are responsible for the constant production and release of leptin. This can also be produced by the placenta. Leptin levels rise during pregnancy and fall after parturition (childbirth). Leptin is also expressed in fetal membranes and the uterine tissue. Uterine contractions are inhibited by leptin. Professor Cappuccio of the University of Warwick has recently discovered that short sleep duration may lead to obesity through an increase of appetite via hormonal changes. Lack of sleep produces ghrelin, a hormone that stimulates appetite by lowering leptin levels.

Next to a biomarker for body fat, serum leptin levels also reflect individual energy balance. Several studies have shown that fasting or following a very low calorie diet (VLCD) lowers leptin levels. It might be that on short-term, leptin is an indicator of energy balance. This system is more sensitive to starvation than to overfeeding, i.e. leptin levels do not rise extensively after overfeeding. It might be that the dynamics of leptin due to an acute change in energy balance are related to appetite and eventually to food intake. Although this is a new hypothesis, there is already some data that supports it.

There is some recognition that leptin action is more decentralized than previously assumed. In addition to its endocrine action at a distance (from adipose tissue to brain), leptin also acts as a paracrine mediator. In fetal lung leptin is induced in the alveolar interstitial fibroblasts (“lipofibroblasts”) by the action of PTHrP secreted by formative alveolar epithelium (endoderm) under moderate stretch. The leptin from the mesenchyme in turn acts back on the epithelium at the leptin receptor carried in the alveolar type II pneumocytes and induces surfactant expression which is one of the main functions of these type II pneumocytes. In addition to white adipose tissue—the major source of leptin—it can also be produced by brown adipose tissue, placenta (syncytiotrophoblasts), ovaries, skeletal muscle, stomach (lower part of fundic glands), mammary epithelial cells, bone marrow, pituitary and liver.

### III. LEPTIN EXPRESSION CONSTRUCTS

A. Promoters

Recombinant vectors that express leptin or a leptin agonist form an important aspect of the present invention. The term “expression vector” or “expression construct” means any type of genetic construct containing a nucleic acid coding for leptin or a leptin agonist that is capable of being transcribed. The leptin-coding region is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of leptin or a leptin agonist.

The promoter may be in the form of a promoter that is naturally-associated with leptin. In other embodiments, it is contemplated that certain advantages will be gained by positioning the leptin coding segment under the control of a recombinant, or heterologous, promoter. As used herein, a recombinant or heterologous promoter is intended to refer to a promoter that is not normally associated with leptin in its natural environment. Such promoters may include promoters normally associated with other genes, and/or promoters isolated from any other bacterial, viral, eukaryotic, or mammalian cells.

Naturally, it will be important to employ a promoter that effectively directs the expression of the DNA segment in the cell/tissue type chosen for expression. The use of promoter and cell type combinations for protein expression is generally known to those of skill in the art of molecular biology, for example, see Sambrook et al. (1989), incorporated herein by reference. The promoters employed may be constitutive, or inducible, and can be used under the appropriate conditions to direct high level expression of leptin or a leptin agonist.

At least one module in a promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

Promoters can be classified into two groups, ubiquitous and tissue- or cell-specific. Ubiquitous promoters activate transcription in all or most tissues and cell types. Examples of ubiquitous promoters are cellular promoters like the histone promoters, promoters for many metabolic enzyme genes such as hexokinase I and glyceraldehyde-3-phosphate dehydrogenase, and many viral promoters such as the cytomegalovirus promoter (CMVp) and the Rous sarcoma virus promoter (RSVp). In certain aspects of the present invention, these promoters are appropriate for use with the immortalizing constructs described herein, as well as finding use in additional aspects of the present invention.

Tissue- or cell-specific promoters activate transcription in a restricted set of tissues or cell types or, in some cases, only in a single cell type of a particular tissue. Examples of stringent cell-specific promoters are the insulin gene promoters which are expressed in only a single cell type (pancreatic β-cells) while remaining silent in all other cell types, and the immunoglobulin gene promoters which are expressed only in cell types of the immune system. The promoter may also be “context specific” in that it will be expressed only in the desired cell type and not in other cell types that are likely to be present in the population of target cells.

Promoters can be modified in a number of ways to increase their transcriptional activity. Multiple copies of a given promoter can be linked in tandem, mutations which increase activity may be introduced, single or multiple copies of individual promoter elements may be attached, parts of unrelated promoters may be fused together, or some combination of all of the above can be employed to generate highly active promoters. All such methods are contemplated for use in connection with the present invention.

Ultimately, the particular promoter that is employed to control the expression of a nucleic acid is not believed to be critical, so long as it is capable of expressing the nucleic acid in the targeted cell. In various other embodiments, the human cytomegalovirus (CMV) immediate early gene promoter, the SV40 early promoter and the Rous sarcoma virus long terminal repeat can be used to obtain high-level expression of transgenes. The use of other human, viral or mammalian cellular promoters which are well-known in the art to achieve expression of a transgene is contemplated as well, provided that the levels of expression are sufficient for a given purpose.

B. Enhancers and Other Elements

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression of a transgene. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Turning to expression, once a suitable clone or clones have been obtained, whether they be cDNA based or genomic, one may proceed to prepare an expression system. The engineering of DNA segment(s) for expression in human neuroendocrine cells may be performed by techniques generally known to those of skill in recombinant expression. It is believed that a number of different expression systems may be employed in the expression of proteins and peptides in the present invention.

In expression, one will also typically desire to incorporate into the transcriptional unit an appropriate polyadenylation site (e.g., 5′-AATAAA-3′) if one was not contained within the original cloned segment. Typically, the poly A addition site is placed about 30 to 2000 nucleotides “downstream” of the termination site of the protein at a position prior to transcription termination. The nature of the polyadenylation signal is not believed to be crucial to the successful practice of the invention, and any such sequence may be employed. Particular embodiments include the SV40 polyadenylation signal and the bovine growth hormone polyadenylation signal, convenient and known to function well in various target cells. Also contemplated as an element of the expression cassette is a terminator. These elements can serve to enhance message levels and to minimize read through from the cassette into other sequences.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

C. Expression Vectors

Expression vectors for use in mammalian such cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., polyoma, adeno, VSV, BPV) source, or may be provided by the host cell chromosomal replication mechanism. If the vector is integrated into the host cell chromosome, the latter is often sufficient.

A number of viral based expression systems may be utilized, for example, commonly used promoters are derived from polyoma, Adenovirus 2, and most frequently Simian Virus 40 (SV40). The early and late promoters of SV40 virus are particularly useful because both are obtained easily from the virus as a fragment which also contains the SV40 viral origin of replication. Smaller or larger SV40 fragments may also be used, provided there is included the approximately 250 bp sequence extending from the HindIII site toward the BglI site located in the viral origin of replication.

1. Non-Viral Delivery

In certain embodiments of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an expression construct complexed with Lipofectamine (Gibco BRL).

Liposome-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of liposome-mediated delivery and expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. Melloul et al. (1993) demonstrated transfection of both rat and human islet cells using liposomes made from the cationic lipid DOTAP, and Gainer et al. (1996) transfected mouse islets using Lipofectamine-DNA complexes.

Another embodiment of the invention for transferring one or more naked DNA immortalizing or other expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

In certain embodiments of the present invention, the expression construct is introduced into the cell using adenovirus assisted transfection. Increased transfection efficiencies have been reported in cell systems using adenovirus coupled systems (Kelleher and Vos, 1994; Cotten et al., 1992; Curiel, 1994), and the inventor contemplates using the same technique to increase transfection efficiencies into human islets.

Still further constructs that may be employed to deliver the one or more immortalizing or other expression construct to the target cells are receptor-mediated delivery vehicles. These take advantage of the selective uptake of macromolecules by receptor-mediated endocytosis that will be occurring in the target cells. In view of the cell type-specific distribution of various receptors, this delivery method adds another degree of specificity to the present invention. Specific delivery in the context of another mammalian cell type is described by Wu and Wu (1993; incorporated herein by reference).

Certain receptor-mediated gene targeting vehicles comprise a cell receptor-specific ligand and a DNA-binding agent. Others comprise a cell receptor-specific ligand to which the DNA construct to be delivered has been operatively attached. Several ligands have been used for receptor-mediated gene transfer (Wu and Wu, 1987, 1988; Wagner et al., 1990; Ferkol et al., 1993; Perales et al., 1994; Myers, EPO 0273085), which establishes the operability of the technique. In the context of the present invention, the ligand will be chosen to correspond to a receptor specifically expressed on the neuroendocrine target cell population.

In other embodiments, the DNA delivery vehicle component of a cell-specific gene targeting vehicle may comprise a specific binding ligand in combination with a liposome. The nucleic acids to be delivered are housed within the liposome and the specific binding ligand is functionally incorporated into the liposome membrane. The liposome will thus specifically bind to the receptors of the target cell and deliver the contents to the cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the DNA delivery vehicle component of the targeted delivery vehicles may be a liposome itself, which will preferably comprise one or more lipids or glycoproteins that direct cell-specific binding. For example, Nicolau et al. (1987) employed lactosyl-ceramide, a galactose-terminal asialganglioside, incorporated into liposomes and observed an increase in the uptake of the insulin gene by hepatocytes. It is contemplated that the one or more immortalizing or other expression constructs of the present invention can be specifically delivered into the target cells in a similar manner.

2. Viral Delivery

One of the preferred methods for delivery of expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue-specific transforming construct that has been cloned therein.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNA's issued from this promoter possess a 5′-tripartite leader (TPL) sequence which makes them preferred mRNA's for translation.

In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the E3, or both the E1 and E3 regions (Graham and Prevec, 1991). In nature, adenovirus can package approximately 105% of the wild-type genome (Ghosh-Choudhury et al., 1987), providing capacity for about 2 kb of extra DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of media. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of media, is added to the carrier (50 ml) in a 250 ml Erlenmeyer flask and left stationary, with occasional agitation, for 1 to 4 h. The media is then replaced with 50 ml of fresh media and shaking initiated. For virus production, cells are allowed to grow to about 80% confluence, after which time the media is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector preferred for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

Recombinant adenovirus and adeno-associated virus (see below) can both infect and transduce non-dividing human primary cells. Gene transfer efficiencies of approximately 70% for isolated rat islets have been demonstrated by the inventor (Becker et al., 1994a; Becker et al., 1994b; Becker et al., 1996) as well as by other investigators (Gainer et al., 1996).

Adeno-associated virus (AAV) is an attractive vector system for use in the human cell transformation of the present invention as it has a high frequency of integration and it can infect nondividing cells, thus making it useful for delivery of genes into mammalian cells in tissue culture (Muzyczka, 1992). AAV has a broad host range for infectivity (Tratschin, et al., 1984; Laughlin, et al., 1986; Lebkowski, et al., 1988; McLaughlin, et al., 1988), which means it is applicable for use with human neuroendocrine cells, however, the tissue-specific promoter aspect of the present invention will ensure specific expression of the transforming construct in aspects of the invention where this is desired or required. Details concerning the generation and use of rAAV vectors are described in U.S. Pat. Nos. 5,139,941 and 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt, et al., 1994; Lebkowski, et al., 1988; Samulski, et al., 1989; Shelling and Smith, 1994; Yoder, et al., 1994; Zhou, et al., 1994; Hermonat and Muzyczka, 1984; Tratschin, et al., 1985; McLaughlin, et al., 1988) and genes involved in human diseases (Flotte, et al., 1992; Luo, et al., 1994; Ohi, et al., 1990; Walsh, et al., 1994; Wei, et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski, et al., 1989; McLaughlin, et al., 1988; Kotin, et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte et al., 1995).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975). Additional retroviral vectors contemplated for use in the present invention have been described (Osborne et al., 1990; Flowers et al., 1990; Stockschlaeder et al., 1991; Kiem et al., 1994; Bauer et al., 1995, Miller and Rosman, 1989; Miller et al., 1993; each incorporated herein by reference).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990). A preferred cell line is the PA317 cell line (Osborne et al., 1990).

A major determinant of virus titer is the number of packagable RNA transcripts per producer cell, which is dependent on the integrated proviral DNA copy number. Packaging cell lines are coated with viral envelope glycoproteins and are thus resistant to infection by virus of the same host range, but not virus of a different host range. This process is called interference. Therefore, recombinant retroviruses can shuttle back and forth between amphotropic and ecotropic packaging cell lines in a mixed culture (referred to as ping-ponging), thus leading to an increase in proviral DNA copy number and virus titer (Bestwick et al., 1988). Some drawbacks to the ping-pong process are that transfer of packaging functions between ecotropic and anphotropic lines can lead eventually to generation of replication-competent helper virus. Also, increasing numbers of cells express both ecotropic and amphotropic envelope proteins and are therefore resistant to further infection. Moreover, cells with large numbers of proviruses are unhealthy. Thus, there is an optimum period during the ping-pong process when virus titer is high and helper virus is absent. This time period is empirically determined and is relatively constant for a given ecotropic plus amphotropic packaging line combination.

Other viral vectors may be employed as expression constructs in the present invention. Vectors derived from viruses such as vaccinia virus (Ridgeway, 1988; Baichwal and Sugden, 1986; Coupar et al., 1988) and herpesviruses may be employed. They offer several attractive features for various mammalian cells (Friedmann, 1989; Ridgeway, 1988; Baichwal and Sugden, 1986; Coupar et al., 1988; Horwich et al., 1990). Lentivirus vectors are also contemplated for use in the present invention (Gallichan et al., 1998; Miyoshi et al., 1998; Kafri et al., 1999).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

### IV. LEPTIN (OB) POLYPEPTIDES AND LEPTIN AGONISTS, ANALOGS, AND DERIVATIVES THEREOF

The terms “protein,” which refers to a polypeptide, and “polypeptide” is used herein interchangeably with respect to a proteinacious portion of a leptin, a leptin agonist, or a leptin derivative, and variants or fragments thereof. Unless otherwise stated, the terms “polypeptide,” “mature protein” and “mature polypeptide” refer to a proteinacious portion of a leptin, a leptin agonist, or a leptin derivative, and variants thereof, and may have the leptin signal sequence (or a fusion protein partner) removed.

In certain embodiments, an exemplary leptin that is employed in the disclosed methods is mature, recombinant methionyl human leptin, which is also known as metreleptin, or rmetHu-Leptin, which has the amino acid sequence, as set forth in one-letter code:

(SEQ ID NO: 13)
MVPIQKVQDDTKTLIKTIVTRINDISHTQSVSSKQKVTGLDFIPGLHPI
LTLSKMDQTLAVYQQILTSMPSRNVIQISNDLENLRDLLHVLAFSKSCH
LPWASGLETLDSLGGVLEASGYSTEVVALSRLQGSLQDMLWQLDLSPGC

In other embodiments, an exemplary leptin, which has 146 amino acids, and, as compared to metreleptin, has a glutamine absent at position 28, presented below (in one-letter code), wherein the blank (“*”) indicates no amino acid):

(SEQ ID NO: 14)
MVPIQKVQDDTKTLIKTIVTRINDISHT*SVSSKQKVTGLDFIPGLHPILTLSKMDQTLA
VYQQILTSMPSRNVIQISNDLENLRDLLHVLAFSKSCHLPWASGLETLDSLGGVLEAS
GYSTEVVALSRLQGSLQDMLWQLDLSPGC

Additionally, a mouse leptin ortholog may be selected for use in the disclosed methods, which mouse leptin ortholog is represented by the primary amino acid sequence indicated below (in three-letter code):

(SEQ ID NO: 15)
Met Val Pro Ile Gln Lys Val Gln Asp Asp Thr Lys Thr Leu Ile Lys Thr Ile Val Thr Arg Ile
Asn Asp Ile Ser His Thr Gln Ser Val Ser Ala Lys Gln Arg Val Thr Gly Leu Asp Phe Ile Pro
Gly Leu His Pro Ile Leu Ser Leu Ser Lys Met Asp Gln Thr Leu Ala Val Tyr Gln Gln Val
Leu Thr Ser Leu Pro Ser Gln Asn Val Leu Gln Ile Ala Asn Asp Leu Glu Asn Leu Arg Asp
Leu Leu His Leu Leu Ala Phe Ser Lys Ser Cys Ser Leu Pro Gln Thr Ser Gly Leu Gln Lys Pro
Glu Ser Leu Asp Gly Val Leu Glu Ala Ser Leu Tyr Ser Thr Glu Val Val Ala Leu Ser Arg
Leu Gln Gly Ser Leu Gln Asp Ile Leu Gln Gln Leu Asp Val Ser Pro Glu Cys

The present invention also contemplates the use of leptin agonists and nucleic acids encoding leptin agonists. A leptin agonist is a molecule that induces the expression of leptin, stabilizes or enhances the activity of leptin, or acts as a mimic of leptin.

As noted above, in specific embodiments a leptin, a leptin agonist, or a leptin derivative, includes those having the amino acid sequences set forth herein, e.g., SEQ ID NOS: 13, 14, 15, etc., including such polypeptides that have been modified with conservative amino acid substitutions, as well as biologically active fragments, analogs, and derivatives thereof. The term “biologically active,” is used herein to refer to a specific effect of the polypeptide, including but not limited to specific binding, e.g., to a receptor, antibody, or other recognition molecule; activation of signal transduction pathways on a molecular level; and/or induction (or inhibition by antagonists) of physiological effects mediated by the native ob polypeptide in vivo. In certain embodiments, “biologically active” leptins, leptin agonists, or leptin derivatives, or “biological activity” of leptins, leptin agonists, or leptin derivatives, refers to, for example, the capacity of such to: treat, reduce, alleviate, suppress attenuate of inhibit type I diabetes or a clinical manifestation thereof; restore normoglycemia; reduce HbAlc; reduce, suppress, attenuate, or inhibit hyperglucogonemia or a condition associated with hyperglucogonemia; reduce, suppress, attenuate, or inhibit excess gluconeogenesis, excess glycogenolysis, hyperglycemia, hyperglucagonemia, ketosis, diabetic ketoacidosis, hypertriglyceridemia, elevated plasma free fatty acid, weight loss, catabolic syndrome, terminal illness, hypertension, diabetic nephropathy, renal insufficiency, renal failure, hyperphagia, muscle wasting, diabetic neuropathy, diabetic retinopathy, or diabetic coma; in a subject diagnosed with or otherwise having type I diabetes, to which such a leptin, leptin analog, or leptin derivative has been provided.

As disclosed, for example in WO 96/05309 and U.S. Pat. No. 5,935,810, murine leptin and human leptin share 83% identity, and human leptin is active in murine. Additionally, as described below, human leptin is active in monkey, which also displays approximately 80% identity to human leptin. Accordingly, leptin proteins, leptin analogs, or leptin derivatives that possess 83% amino acid identity or greater, including, for example, 83% amino acid identity, 84% amino acid identity, 85% amino acid identity, 86% amino acid identity, 87% amino acid identity, 88% amino acid identity, 89% amino acid identity, 90% amino acid identity, 91% amino acid identity, 92% amino acid identity, 93% amino acid identity, 94% amino acid identity, 95% amino acid identity, 96% amino acid identity, 97% amino acid identity, 98% amino acid identity, or 99% amino acid identity, may be employed in accordance with the disclosed and claimed methods.

Leptin polypeptides, including fragments, analogs, and derivatives, can be prepared synthetically, e.g., using the well known techniques of solid phase or solution phase peptide synthesis. Solid phase synthetic techniques may be employed. Alternatively, such leptin polypeptides can be prepared using well known genetic engineering techniques.

In certain embodiments, naturally occurring fragments of the OB polypeptide may be employed. The peptide sequence includes a number of sites that are frequently the target for proteolytic cleavage, e.g., arginine residues. It is possible that the full length polypeptide may be cleaved at one or more such sites to form biologically active fragments. In certain embodiments, leptin fragments such as those disclosed in U.S. Pat. Nos. 6,777,388, 7,186,694, 7,208,572, and PCT Publication No. WO 2004/039832 may be employed in accordance with the herein disclosed and claimed methods. In certain embodiments the fragment corresponding to amino acids 116-130 of the unprocessed, 167 amino acid leptin protein expressed in humans.

Additionally leptin analogs, which are characterized by, for example, being capable of a biological activity as described above, may be employed in accordance with the disclosed and claimed methods. In certain embodiments, such leptin analogs may be more effective than the native leptin protein. For example, a leptin analog may bind to the OB receptor with higher affinity, or demonstrate a longer half-life in vivo, or both, or display greater efficacy or potency with regard to a biological activity as described above, relative to a native leptin protein. Nevertheless, leptin analogs that are less effective than the native protein are also contemplated.

In one embodiment, an analog of OB peptide is the OB peptide modified by substitution of amino acids at positions on the polypeptide that are not essential for structure or function. For example, since it is known that human OB peptide is biologically active in mouse, substitution of divergent amino acid residues in the human sequence as compared to the murine amino acid sequence will likely yield useful analogs of OB peptide. For example, the serine residue at position 53 or position 98, or both of the unprocessed, 167 amino acid human leptin protein may be substituted, e.g., with glycine, alanine, valine, cysteine, methionine, or threonine. Similarly, the arginine residue at position number 92 may be substituted, e.g., with asparagine, lysine, histidine, glutamine, glutamic acid, aspartic acid, serine, threonine, methionine, or cysteine. Other amino acids in the human leptin protein that are capable of substitution are histidine at position 118, tryptophan at position 121, alanine at position 122, glutamic acid at position 126, threonine at position 127, leucine at position 128, glycine at position 132, glycine at position 139, tryptophan at position 159, and glycine at position 166. In another embodiment, substitution of one or more of residues 121 to 128, e.g., with glycines or alanines, or substituting some of the residues with the exceptions of serine as position 123, or leucine at position 125.

In another embodiment, an analog of the leptin polypeptide, preferably the human leptin polypeptide, is a truncated form of the polypeptide. For example, it has already been demonstrated that the glutamine at residue 49 is not essential, and can be deleted from the peptide. Similarly, it may be possible to delete some or all of the divergent amino acid residues at positions 121-128. In addition, the invention contemplates providing an leptin analog having the minimum amino acid sequence necessary for a biological activity. This can be readily determined, e.g., by testing the activity of fragments of OB for the ability to bind to OB-specific antibodies, inhibit such biological activity of the native human leptin polypeptide, or agonize the activity of the native leptin peptide. In one embodiment, the invention provides a truncated leptin polypeptide consisting of the loop structure formed by the disulfide bond that forms between cysteine residues 117 and 167. In another embodiment, the truncated analog corresponds to the amino acids from residue 22 (which follows the putative signal peptide cleavage site) to 53 (the amino acid residue immediately preceding a flexible loop region detected with limited proteolysis followed by mass spectrometric analysis of the OB polypeptide; see Cohen et al. (1995). In another embodiment, the truncated analog corresponds to amino acids from residue 61 (the residue immediately following the flexible loop region as detected with the limited proteolysis/mass, spec. analysis of the OB polypeptide) to amino acid residue 116 (the residue immediately preceding the first cysteine residue). In yet another embodiment, the truncated analog corresponds to amino acids from residue 61 to amino acid residue 167.

Furthermore, one or more of the residues of the putative flexible loop at residues number 54 to 60 are substituted. For example, one or more of the residues may be substituted with lysine, glutamic acid, or cysteine (such as lysine) for cross linking, e.g., to a polymer, since flexible loop structures are preferred sites for derivatization of a protein. Alternatively, the residues at the flexible loop positions may be substituted with amino acid residues that are more resistant to proteolysis but that retain a flexible structure, e.g., one or more prolines. In yet another embodiment, substitutions with amino acid residues that can be further derivatized to make them more resistant to degradation, e.g., proteolysis, is contemplated.

It will be appreciated by one of ordinary skill in the art that the foregoing fragment sizes are approximate, and that from one to about five amino acids can be included or deleted from each or both ends, or from the interior of the polypeptide or fragments thereof, of the recited truncated analogs, with the exception that in the disulfide bonded loop analogs, the cysteine residues must be maintained.

It has been found that murine leptin polypeptide contains 50% α-helical content, and that the human leptin polypeptide contains about 60% α-helical content, as detected by circular dichroism of the recombinant peptides under nearly physiological conditions.

Accordingly, in another embodiment, amino acid residues can be substituted with residues to form analogs of leptin polypeptide that demonstrate enhanced propensity for forming, or which form more stable, α-helix structures. For example, α-helix structure would be preferred if Glu, Ala, Leu, His, Trp are introduced as substitutes for amino acid residues found in the native OB polypeptide. In particular, conservative amino acid substitutions are employed, e.g., substituting aspartic acid at residue(s) 29, 30, 44, 61, 76, 100, and/or 106 with glutamic acid(s) (Glu); substituting isoleucine(s) with leucine; substituting glycine or valine, or any divergent amino acid, with alanine (e.g., serine at position 53 of the human leptin polypeptide with alanine), substituting arginine or lysine with histidine, and substituting tyrosine and/or phenylalanine with tryptophan. Increasing the degree, or more importantly, the stability of α-helix structure may yield a leptin analog with greater activity, increased binding affinity, or longer half-life. In one embodiment, the helix forming potential of the portion of the leptin peptide corresponding to amino acid residues 22 through 53 is increased. In another embodiment, the helix-forming potential or stability of the amino acid residues 61-116 is increased. In yet another embodiment, the helix forming potential of the disulfide loop structure corresponding to amino acids 117 to 167 is increased. Also contemplated are leptin analogs containing enhanced α-helical potential or stability in more than one of the foregoing domains. In another embodiment, truncated leptin polypeptide analogs are generated that incorporate structure-forming, e.g., helix forming, amino acid residues to compensate for the greater propensity of polypeptide fragments to lack stable structure.

Analogs, such as fragments, may be produced, for example, by pepsin digestion of weight modulator peptide material. Other analogs, such as muteins, can be produced by standard site-directed mutagenesis of weight modulator peptide coding sequences.

The murine protein is substantially homologous to the human protein, particularly as a mature protein, and, further, particularly at the N-terminus. One may prepare an analog of the recombinant human protein by altering (such as substituting amino acid residues), in the recombinant human sequence, the amino acids which diverge from the murine sequence. Because the recombinant human protein has biological activity in mice (see, e.g., WO 98/28427, WO 96/05309, U.S. Pat. Nos. 6,429,290, 5,935,810, 6,001,968, herein incorporated by reference in their entirety) such an analog would likely be active in accordance with the herein disclosed and claimed methods, in humans.

For example, using a human protein having a lysine at residue 36 and an isoleucine at residue 75 according to the numbering of SEQ. ID. NO. 13, 14, or 15, wherein the first amino acid is a methionine, and the amino acid at position 147 is cysteine, one may substitute with another amino acid one or more of the amino acids at positions 33, 36, 51, 65, 69, 72, 75, 78, 90, 98, 101, 106, 107, 108, 109, 112, 119, 137, 139, 143, and 146. One may select the amino acid at the corresponding position of the murine protein, (SEQ. ID. NO. 15), or another amino acid. In certain embodiments a conservative change is effected at one or more of such positions by substituting an amino acid with similar physio-chemical properties for a given amino acid at such position, as is understood in the art.

One may further prepare “consensus” molecules based on the rat leptin protein sequence. Murakami et al. (1995) and WO 98/28427, both herein incorporated by reference in their entirety. Rat leptin protein differs from human leptin protein at the following positions of the human leptin mature protein (SEQ ID NO: 13): 5, 33, 34, 36, 51, 69, 72, 75, 78, 79, 90, 98, 101, 102, 103, 106, 107, 108, 109, 112, 119, 137, 139 and 146. One may substitute with another amino acid one or more of the amino acids at these divergent positions. At one or more of such positions, one may effect a substitution of an amino acid from the corresponding rat leptin protein, or another amino acid. In certain embodiments a conservative change is effected at one or more of such positions by substituting an amino acid with similar physio-chemical properties for a given amino acid at such position, as is understood in the art.

The positions from both rat and murine leptin protein which diverge from the mature human leptin protein as set out in SEQ ID NO:13 are: 5, 33, 34, 36, 51, 65, 69, 72, 75, 78, 79, 90, 98, 101, 103, 105, 107, 108, 109, 112, 119, 137, 139, 143, and 146 (see, e.g., WO 98/28427). Accordingly, a leptin protein according to SEQ. ID. NO. 13, 14, or 15, having one or more amino acids at the above positions replaced with another amino acid, such as the amino acid found in SEQ ID NO: 14 or 15, 13 or 15, or 13 and 14, respectively may be employed in the disclosed and claimed methods. In addition, the amino acids found in rhesus monkey leptin protein which diverge from the mature human leptin protein are (with identities noted in parentheses in one letter amino acid abbreviation): 9 (S), 36 (R), 49(V), 54(O), 61(1), 67(1), 68(N), 69((L), 90(L), 101(L), 109(E), 113 (D), and 119 (L). Since the recombinant human leptin protein is active in cynomolgus monkeys (see, e.g., WO 97/018833, incorporated herein by reference in its entirety), a human leptin protein according to SEQ. ID. NO. 13 (with lysine at position 36 and isoleucine at position 75) having one or more of the rhesus monkey divergent amino acids replaced with another amino acid, such as the amino acids in parentheses, may be employed in the practice of the disclosed and claimed methods. It should be noted that certain rhesus divergent amino acids are also those found in the above murine species (positions 36, 69, 90, 101 and 113). Thus, one may prepare a murine/rhesus/human consensus molecule having (using the numbering of SEQ. ID. NO. 13 having a lysine at position 36 and an isoleucine at position 75) having one or more of the amino acids at positions replaced by another amino acid: 5, 9, 33, 34, 36, 49, 51, 54, 61, 65, 67, 68, 69, 72, 75, 78, 79, 90, 98, 101, 103, 106, 107, 108, 109, 112, 113, 119, 137, 139, 143, and 146.

Other analogs may be prepared by deleting a part of the protein amino acid sequence. For example, the mature protein lacks an N-terminal leader sequence, also known as a signal sequence, as disclosed, for example, in WO 96/05309, incorporated by reference in its entirety. One may prepare the following truncated forms of human leptin protein molecules (using the numbering of SEQ. ID. NO:13: (a) amino acids 99-147 (b) amino acids 1-33 (c) amino acids 41-117 (d) amino acids 1-100 and (connected to) 113-147 (e) amino acids 1-100 and (connected to) 113-147 having one or more of amino acids 101-112 placed between amino acids 100 and 113. In addition, the truncated forms may also have altered one or more of the amino acids which are divergent (in the rat, murine, or rhesus leptin protein) from human leptin protein. Furthermore, any alterations may be in the form of altered amino acids, such as peptidomimetics or D-amino acids.

The present disclosure provides means of inducing hyperleptinemia and or effect a sustained elevated serum leptin level for a prolonged period of time, for example a few to several days, weeks, or months, such means including viral leptin gene delivery and resulting leptin protein expression and by continuous leptin protein administration by subcuteous pump delivery. Accordingly, leptin proteins, leptin protein analogs, and leptin protein derivatives that possess extended circulation half-life, resistance to protein or protease degradation, and/or display attenuated rates of clearance from the blood may be employed in accordance with the disclosed and claimed methods. Thus, for example, fusion protein partners may be attached, either by recombinant means or by chemical conjugation, to the herein-described and incorporated leptin proteins, leptin analogs, and leptin derivatives. Exemplary such partners include: for example, one or more immunoglobulin heavy chain moeities, e.g., Fc regions; one or more Fab regions; one or more albumins, one or more circulating serum proteins, such as one or more ployaminoacid polymers; one or more small peptide tags, such as His-tags, and the like.

In certain embodiments, Fc-leptin fusion proteins, wherein a leptin protein, a leptin analog, or a leptin derivative as described herein is selected from: (a) the amino acid sequence 1-147 as set forth in SEQ. ID. NO. 13, 14, or 15, or such sequences having a lysine residue at position 36 and an isoleucine residue at position 75; (c) the amino acid sequence of subpart (b) having a different amino acid substituted in one or more of the following positions (using the numbering according to SEQ. ID. NO. 13 and retaining the same numbering even in the absence of a glutaminyl residue at position 28): 5, 33, 34, 36, 51, 65, 69, 72, 75, 78, 79, 90, 98, 101, 103, 106, 107, 108, 109, 112, 119, 137, 139, 143, and 146; (d) the amino acid sequence of subparts (a), (b) or (c) optionally lacking a glutaminyl residue at position 28; (e) a truncated leptin protein analog selected from among: (using the numbering of SEQ. ID. NO. 13): (i) amino acids 99-147 (ii) amino acids 1-33 (iii) amino acids 40-117 (iv) amino acids 1-100 and 113-147 (v) amino acids 1-100 and 113-147 having one or more of amino acids 101-112 placed between amino acids 100 and 113; and, (vi) the truncated leptin analog of subpart (i) having one or more of amino acids 101, 103, 106, 107, 108, 109, 112, 119, 137, 139, 143, and substituted with another amino acid; (vii) the truncated analog of subpart (ii) having one or more of amino acids 5, 9 and 33 substituted with another amino acid; (viii) the truncated analog of subpart (iii) having one or more of amino acids 51, 54, 61, 65, 67, 68, 69, 72, 75, 78, 79, 90, 98, 101, 103, 106, 107, 108, 109, 112 and 113 replaced with another amino acid; (vix) the truncated analog of subpart (iv) having one or more of amino acids 5, 9, 33, 34, 36, 49, 51, 54, 61, 65, 67, 68, 69, 72, 75, 78, 79, 90, 98, 113, 119, 137, 139, 143, and 146 replaced with another amino acid; and (x) the truncated analog of subpart (v) having one or more of amino acids 5, 33, 34, 36, 51, 65, 69, 72, 75, 78, 79, 90, 98, 101, 103, 106, 107, 108, 109, 112, 119, 137, 139, 143, and 146 replaced with another amino acid; and (g) the leptin protein or analog derivative of any of subparts (a) through (f) comprised of a chemical moiety connected to the protein moiety; (h) a derivative of subpart (g) wherein said chemical moiety is a water soluble polymer moiety; (i) a derivative of subpart (h) wherein said water soluble polymer moiety is polyethylene glycol; (j) a derivative of subpart (h) wherein said water soluble polymer moiety is a polyaminoacid moiety; (k) a derivative of subpart (h) through (j) wherein said moiety is attached at solely the N-terminus of said protein moiety; and (1) an OB protein, analog or derivative of any of subparts (a) through (k) in a pharmaceutically acceptable carrier.

In certain embodiments, a leptin protein, leptin analog, or leptin derivative may be prepared by attachment of one or more chemical moieties to such. Such chemically modified proteins, analogs, or derivatives may be further formulated for intraarterial, intraperitoneal, intramuscular subcutaneous, intravenous, oral, nasal, pulmonary, topical or other routes of administration as discussed below. Chemical modification of biologically active proteins has been found to provide additional advantages under certain circumstances, such as increasing the stability and circulation time of the therapeutic protein and decreasing immunogenicity. See U.S. Pat. No. 4,179,337; for a review, see Abuchowski et al. (1981).

The chemical moieties suitable for such derivatization may be selected from among various water soluble polymers. The polymer selected should be water soluble so that the protein to which it is attached does not precipitate in an aqueous environment, such as a physiological environment. Preferably, for therapeutic use of the end-product preparation, the polymer will be pharmaceutically acceptable. one skilled in the art will be able to select the desired polymer based on such considerations as whether the polymer/protein conjugate will be used therapeutically, and if so, the desired dosage, circulation time, resistance to proteolysis, and other considerations. For the present proteins and peptides, the effectiveness of the derivatization may be ascertained by administering the derivative, in the desired form (i.e., by osmotic pump, or, more preferably, by injection or infusion, or, further formulated for oral, pulmonary or nasal delivery, for example), and observing biological effects as described herein. The water soluble polymer may be selected from the group consisting of, for example, polyethylene glycol, copolymers of ethylene glycol/propylene glycol, carboxymethylcellulose, dextran, polyvinyl alcohol, polyvinyl pyrolidone, poly-1,3-dioxolane, poly-1,3,6-trioxane, ethylene/maleic anhydride copolymer, polyaminoacids (either homopolymers or random copolymers), and dextran or poly(n-vinyl pyrolidone)polyethylene glycol, propylene glycol homopolymers, polypropylene oxide/ethylene oxide co-polymers, polyoxyethylated polyols and polyvinyl alcohol. Polyethylene glycol propionaldenhyde may have advantages in manufacturing due to its stability in water. Also, succinate and styrene may also be used.

The derivatized leptin, leptin analog, or leptin derivative, may be prepared by attaching polyamino acids or branch point amino acids to the leptin moiety (e.g., protein, analog, or derivative) which, serves to also increase the circulation half life of the protein in addition to the advantages achieved via the modifications described above.

For the therapeutic purpose of the present invention, such polyaminoacids should be those which have or do not create neutralizing antigenic response, or other adverse responses. Such polyaminoacids may be selected from the group consisting of serum album (such as human serum albumin), an additional antibody or portion thereof (e.g., the Fc region), or other polyaminoacids, e.g., lysines. As indicated below, the location of attachment of the polyaminoacid may be at the N-terminus of the leptin protein moiety, or C-terminus, or other places in between, and also may be connected by a chemical “linker” moiety to the leptin moiety.

A polymer which may be attached to a leptin protein, leptin analog, or leptin derivative may be of any molecular weight, and may be branched or unbranched. For polyethylene glycol, a particular molecular weight is between about 2 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The number of polymer molecules so attached may vary, and one skilled in the art will be able to ascertain the effect on function. One may mono-derivatize, or may provide for a di-, tri-, tetra or some combination of derivatization, with the same or different chemical moieties (e.g., polymers, such as different weights of polyethylene glycols). The proportion of polymer molecules to protein (or peptide) molecules will vary, as will their concentrations in the reaction mixture. In general, the optimum ratio (in terms of efficiency of reaction in that there is no excess unreacted protein or polymer) will be determined by factors such as the desired degree of derivatization (e.g., mono, di-, tri-, etc.), the molecular weight of the polymer selected, whether the polymer is branched or unbranched, and the reaction conditions.

The chemical moieties should be attached to the protein with consideration of effects on functional or antigenic domains of the protein. There are a number of attachment methods available to those skilled in the art, e.g., EP 0 401 384 herein incorporated by reference (coupling PEG to G-CSF), see also Malik et al. (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residue. Those having a free carboxyl group may include aspartic acid residues, glutamic acid residues, and the C-terminal amino acid residue. Sulfhydryl groups may also be used as a reactive group for attaching the polyethylene glycol molecule(s). For therapeutic purposes one many attach at an amino group, such as attachment at the N-terminus or lysine group. Attachment at residues important for receptor binding should be avoided if receptor binding is desired.

One may specifically desire N-terminally chemically-modified leptin moiety fusion protein. Using polyethylene glycol as an illustration of the present compositions, one may select from a variety of polyethylene glycol molecules (by molecular weight, branching, etc.), the proportion of polyethylene glycol molecules to protein (or peptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective N-terminal chemical modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved. For example, one may selectively N-terminally pegylate the protein by performing the reaction at a pH which allows one to take advantage of the pKa differences between the e-amino group of the lysine residues and that of the a-amino group of the N-terminal residue of the protein. By such selective derivatization, attachment of a water soluble polymer to a protein is controlled: the conjugation with the polymer takes place predominantly at the N-terminus of the protein and no significant modification of other reactive groups, such as the lysine side chain amino groups, occurs. Using reductive alkylation, the water soluble polymer may be of the type described above, and should have a single reactive aldehyde for coupling to the protein. Polyethylene glycol propionaldehyde, containing a single reactive aldehyde, may be used.

An N-terminally monopegylated derivative is preferred for ease in production of a therapeutic.

N-terminal pegylation ensures a homogenous product as characterization of the product is simplified relative to di-, tri- or other multi-pegylated products. The use of the above reductive alkylation process for preparation of an N-terminal product is preferred for ease in commercial manufacturing.

Additionally, acetylation of the leptin proteins, leptin analogs, and leptin derivatives disclosed herein may be employed.

Additional Leptin protein and leptin protein containing compositions appropriate for use in the methods and compositions described herein are known in the art and include, but are not limited to a pegylated (PEG) leptin protein such as the PEG-leptin disclosed by Hoffman La Roche. Other leptin proteins, analogs, derivatives, preparations, formulations, pharmaceutical compositions, doses, and administration routes have previously been described in the following patent publications and are hereby incorporated by reference in their entirety and for all purposes: U.S. Pat. Nos. 5,552,524; 5,552,523; 5,552,522; 5,521,283, 5,935,810; 6,001,968; 6,429,290; 6,350,730; 6,936,439; 6,420,339; 6,541,033; U.S. Patent Publications U.S. 2004/0072219, 2003/049693, 2003/0166847, 2003/0092126, 2005/0176107; 2005/0163799; and PCT Application Publications WO 96/05309, WO 96/40912; WO 97/06816, WO 00/20872, WO 97/18833, WO 97/38014, WO 98/08512, WO 98/28427, WO 98/46257, WO 00/09165, WO 00/47741, and WO 00/21574, and U.S. Pat. Nos. 6,777,388 and 6,936,439. Means for testing for leptin agonism or antagonism are described, e.g., in U.S. Pat. Nos. 6,007,998 and 5,856,098. These patents are exemplary and are incorporated herein by reference in their entirety and for all purposes.

Additional leptin proteins, leptin agonists, leptin derivatives, and fragments thereof, and preparations, formulations, pharmaceutical compositions, doses, administration dosages, rates, and routes of administration of such, have previously been described in the following patent publications and are hereby incorporated by reference in their entirety and for all purposes: U.S. Pat. Nos. 5,552,524, 5,552,523, 5,552,522, 5,521,283, 5,935,810, 6,001,968, 6,429,290, 6,350,730, 6,936,439, 6,420,339, 6,541,033, 6,777,388, 5,525,705, 5,532,336, 5,554,727, 5,563,243 5,559,208, 5,563,243, 5,563,244, 5,563,245, 5,567,678, 5,567,803, 5,569,743, 5,569,744, 5,574,133, 5,580,954, 5,594,101, 5,594,104, 5,605,886, 5,614,379, 5,691,309, 5,719,266, 5,831,017, 5,840,517, 5,851,995, 5,919,902, 5,972,888, 6,221,838, 6,395,509; U.S. Patent Publications 2005/0176107, 2005/0163799, 2004/0072219, 2003/049693, 2003/0166847, 2003/0092126; and PCT Application Publications WO 96/05309, WO 96/40912; WO 97/06816, WO 00/20872, WO 97/18833, WO 97/38014, WO 98/08512, WO 98/28427, WO 98/46257, WO 00/09165, WO 00/47741, and WO 00/21574, hereby incorporated by reference in their entirety. Means for testing for leptin agonism are described, e.g., in U.S. Pat. Nos. 6,007,998 and 5,856,098.

As mentioned above, U.S. Pat. No. 7,112,659 (incorporated by reference) discloses leptin agonists that are Fc-OB fusion protein compositions. In particular, it relates to the genetic or chemical fusion of the Fc region of immunoglobulins to the N-terminal portion of the OB protein. Fusion of Fc at the N-terminus of the OB protein demonstrated advantages not seen in OB protein or with fusion of Fc at the C-terminus of the OB protein. The N-terminally modified Fc-OB protein provides unexpected protein protection from degradation, increased circulation time and increased stability. Accordingly, the Fc-OB fusion protein, and analogs or derivatives thereof, are useful as leptin agonists.

U.S. Pat. No. 7,208,577 (incorporated by reference) discloses that administration of the OB protein to non-obese as well as obese animals results in an increase of lean tissue mass. It also provides methods of treating diabetes, and reducing the levels of insulin necessary for the treatment of diabetes. The increase in lean tissue mass, with concomitant decrease in fat tissue mass, increases sensitivity to insulin. Therefore, the methods require the use of OB protein, or analogs or derivatives thereof, for the reduction of fat tissue mass in order to decrease the amount of insulin necessary for the treatment of diabetes. Derivatives include fusion proteins, chemically-modified versions (i.e., conjugated to solubilizing entities, stabilized) and formulations thereof.

### V. PHARMACEUTICAL FORMULATIONS, ROUTES AND REGIMES FOR ADMINISTRATION

Where clinical applications are contemplated, it will be necessary to prepare pharmaceutical compositions in a form appropriate for administration to a subject. The compositions will generally be prepared essentially free of pyrogens, as well as other impurities that could be harmful to humans or animals. One will generally desire to employ appropriate salts and buffers to render stable cells suitable for introduction into a patient. Aqueous compositions of the present invention comprise an effective amount of stable cells dispersed in a pharmaceutically acceptable carrier or aqueous medium, and preferably encapsulated.

The phrase “pharmaceutically or pharmacologically acceptable” refer to compositions that do not produce adverse, allergic, or other untoward reactions when administered to an animal or a human. As used herein, “pharmaceutically acceptable carrier” includes any and all solvents, dispersion media, antibacterial and antifungal agents, isotonic and absorption delaying agents and the like. As used herein, this term is particularly intended to include biocompatible implantable devices and encapsulated cell populations. The use of such media and agents for pharmaceutically active substances is well know in the art. Except insofar as any conventional media or agent is incompatible with the compositions of the present invention, its use in therapeutic compositions is contemplated. Supplementary active ingredients also can be incorporated into the compositions.

Under ordinary conditions of storage and use, the cell preparations may further contain a preservative to prevent growth of microorganisms. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

The compositions will advantageously be administered by injection, including intravenously, intradermally, intraarterially, intraperitoneally, intracranially, intraarticularly, intraprostaticaly, intrapleurally, intramuscularly, intrahepatically, subcutaneously, or by other method or any combination of the forgoing as would be known to one of ordinary skill in the art.

As will be recognized by those in the field, a “therapeutically effective amount” of a leptin, leptin agaonis, or leptin derivative refers to an mount of such that, when provided to a subject in accordance with the disclosed and claimed methods effects one of the following biological activities: treats type I diabetes; restores normoglycemia; reduces, suppresses, attenuates, or inhibits hyperglucogonemia or a condition associated with hyperglucogonemia; and reduces HbAlc; in a subject diagnosed with or otherwise having type I diabetes. In certain embodiments, such therapeutically effective amount effects such an activity in a subject that is essentially devoid of endogenous inculin. In other embodiments, such therapeutically effective amount effects such an activity in a subject the absence of the provision of exogenous insulin.

As understood in the art, such therapeutically effective amount will vary with many factors including the age and weight of the patient, the patient's physical condition, the condition to be treated, and other factors. An effective amount of the disclosed leptins, leptin agonists, and leptin derivatives will also vary with the particular combination administered. However, typical doses may contain from a lower limit of about 1 μg, 5 μg, 10 μg, 50 μg to 100 μg to an upper limit of about 100 μg, 500 μg, 1 mg, 5 mg, 10 mg, 50 mg or 100 mg of the pharmaceutical compound per day. Also contemplated are other dose ranges such as 0.1 μg to 1 mg of the compound per dose. The doses per day may be delivered in discrete unit doses, provided continuously in a 24 hour period or any portion of that the 24 hours. The number of doses per day may be from 1 to about 4 per day, although it could be more. Continuous delivery can be in the form of continuous infusions. The terms “QID,” “TID,” “BID” and “QD” refer to administration 4, 3, 2 and 1 times per day, respectively. Exemplary doses and infusion rates include from 0.005 nmol/kg to about 20 nmol/kg per discrete dose or from about 0.01/pmol/kg/min to about 10 pmol/kg/min in a continuous infusion. These doses and infusions can be delivered by intravenous administration (i.v.) or subcutaneous administration (s.c.). Exemplary total dose/delivery of the pharmaceutical composition given i.v. may be about 2 μg to about 8 mg per day, whereas total dose/delivery of the pharmaceutical composition given s.c may be about 6 μg to about 6 mg per day.

The disclosed leptins, leptin analogs, and leptin derivatives may be administered, for example, at a daily dosage of, for example: from about 0.01 mg/kg to about 100 mg/kg; from about 0.01 mg/kg to about 80 mg/kg; from about 0.01 mg/kg to about 70 mg/kg; from about 0.01 mg/kg to about 60 mg/kg; from about 0.01 mg/kg to about 50 mg/kg; from about 0.01 mg/kg to about 40 mg/kg; from about 0.01 mg/kg to about 30 mg/kg; from about 0.01 mg/kg to about 25 mg/kg; from about 0.01 mg/kg to about 20 mg/kg; from about 0.01 mg/kg to about 15 mg/kg; from about 0.01 mg/kg to about 10 mg/kg; from about 0.01 mg/kg to about 5 mg/kg; from about 0.01 mg/kg to about 3 mg/kg; from about 0.01 mg/kg to about 1 mg/kg; from about 0.01 mg/kg to about 0.3 mg/kg from about 100 mg/kg to about 90 mg/kg; from about 100 mg/kg to about 80 mg/kg; from about 100 mg/kg to about 70 mg/kg; from about 100 mg/kg to about 60 mg/kg; from about 100 mg/kg to about 50 mg/kg; from about 100 mg/kg to about 40 mg/kg; from about 85 mg/kg to about 10 mg/kg; from about 75 mg/kg to about 20 mg/kg; from about 65 mg/kg to about 30 mg/kg; from about 55 mg/kg to about 35 mg/kg; or from about 55 mg/kg to about 45 mg/kg. Administration may be by injection of a single dose or in divided doses.

In certain embodiments, the disclosed leptins, leptin analogs, and leptin derivatives are administered in order to achieve hyperleptinemia, or substantially supraphysiologic serum levels relative to leptin levels observed in non-type I diabetic subjects.

In other embodiments, the disclosed leptins, leptin analogs, and leptin derivatives are administered in the form of replacement therapy so as to achieve near physiological concentrations of leptin in the plasma. It is estimated that the physiological replacement dose of leptin is about 0.02 mg/kg of body weight per day for males of all ages, about 0.03 mg/kg of body weight per day for females under 18 years and about 0.04 mg/kg of body weight per day for adult females. When attempting to achieve near physiological concentrations of leptin, one may, for example, treat a subject with 50 percent of the estimated replacement dose for the first month of treatment, 100 percent of the replacement dose for the second month of treatment, 200 percent of the replacement dose for the third month of treatment, etc. Serum leptin levels can be measured by methods known in the art, including, for example, using commercially available immunoassays.

The term “unit dose” refers to a physically discrete unit suitable for use in a subject, each unit containing a predetermined quantity of the composition calculated to produce the desired response in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner and are peculiar to each individual.

In general, eligible patients will be treated with their usual regime of diet and human recombinant leptin for 2 weeks. After a complete stable baseline has been obtained, insulin dose will be reduced by 25% and leptin will be administered at a dose of 0.16 mg/kg body weight/day (in two divided doses) in the female subjects and at a dose of 0.08 mg/kg body weight/day (in two divided doses) in the male subjects. Previous studies in CGL patients, this dose resulted in twice the normal physiological plasma levels of leptin in both females and in males. If glucose levels decline, insulin will be reduced by another 25%. If they do not, or if they rise moderately, the leptin dose will be doubled, and the same evaluation carried out. The goal will be to reduce the insulin dose to 15-25% of its original level, and lower circulating insulin levels to the normal basal range. Patients will generally receive leptin by subcutaneous injections, but other routes may be used (including transportable pump units). They will generally be admitted for baseline evaluation until it is felt that they can be followed no less safely as out-patients. Continuous glucose monitoring will be required until stabilization of glucose and the doses have been reached.

### VI. ADJUNCT THERAPIES AND PROCEDURES

A. Insulin Therapy

In accordance with the present invention, it may prove advantageous to combine the methods disclosed herein with adjunct therapies or procedures to enhance the overall anti-diabetic effect. Such therapies and procedures are set forth in general, below. A skilled physician will be apprised of the most appropriate fashion in which these therapies and procedures may be employed.

The present invention, though designed to eliminate the need for other therapies, is contemplated to provide advantageous use with traditional insulin supplementation, but at lower levels, such as below 90%, below 80%, below 70%, below 60%, below 50%, below 40%, below 30%, below 20%, below 15%, 10-15%, below 10%, 5-10%, below 5%, 4%, 3%, 2% or 1% of the normal daily dosage of insulin. Normal daily dosage for TD1 is 30-60 units per day. Such therapies should be tailored specifically for the individual patient given their current clinical situation, and it is contemplated that a subject could be “weaned” down or off insulin therapy after commencing of leptin or leptin agonist provision. The following are general guidelines for typical a “monotherapy” using insulin supplementation by injection, and can be applied here, albeit in the context of the aforementioned reductions in total daily dosage.

Insulin can be injected in the thighs, abdomen, upper arms or gluteal region. In children, the thighs or the abdomen are preferred. These offer a large area for frequent site rotation and are easily accessible for self-injection. Insulin injected in the abdomen is absorbed rapidly while from the thigh it is absorbed more slowly. Hence, patients should not switch from one area to the other at random. The abdomen should be used for the time of the day when a short interval between injection and meal is desired (usually pre-breakfast when the child may be in a hurry to go to school) and the thigh when the patient can wait 30 minutes after injection for his meal (usually pre-dinner). Within the selected area systematic site rotation must be practiced so that not more than one or two injections a month are given at any single spot. If site rotation is not practiced, fatty lumps known as lipohypertrophy may develop at frequently injected sites. These lumps are cosmetically unacceptable and, what is more important, insulin absorption from these regions is highly erratic.

Before injecting insulin, the selected site should be cleaned with alcohol. Injecting before the spirit evaporates can prove to be quite painful. The syringe is held like a pen in one hand, pinching up the skin between the thumb and index finger of the other hand, and inserting the needle through the skin at an angle of 45-90° to the surface. The piston is pushed down to inject insulin into the subcutaneous space (the space between the skin and muscle), then one waits for a few seconds after which release the pinched up skin before withdrawing the needle. The injection site should not be massaged.

For day-to-day management of diabetes, a combination of short acting and intermediate acting insulin is used. Some children in the first year after onset of diabetes may remain well controlled on a single injection of insulin each day. However, most diabetic children will require 2,3 or even 4 shots of insulin a day for good control. A doctor should decide which regimen is best suited.

One injection regimen: A single injection comprising a mix of short acting and intermediate acting insulin (mixed in the same syringe) in 1:3 or 1:4 proportion is taken 20 to 30 minutes before breakfast. The usual total starting dose is 0.5 to 1.0 units/kg body weight per day. This regimen has three disadvantages: (1) all meals must be consumed at fixed times; (2) since the entire quantity of insulin is given at one time, a single large peak of insulin action is seen during the late and early evening hours making one prone to hypoglycemia at this time; (3) as the action of intermediate acting insulin rarely lasts beyond 16-18 hours, the patient's body remains underinsulinized during the early morning hours, the period during which insulin requirement in the body is actually the highest.

Two-injection regimen: This regimen is fairly popular. Two shots of insulin are taken—one before breakfast (⅔ of the total dose) and the other before dinner (⅓ of the total dose). Each is a combination of short acting and intermediate acting insulin in the ratio of 1:2 or 1:3 for the morning dose, and 1:2 or 1:1 for the evening dose. With this regimen the disadvantages of the single injection regimen are partly rectified. Some flexibility is possible for the evening meal. Further, as the total days' insulin is split, single large peaks of insulin action do not occur hence risk of hypoglycemia is reduced and one remains more or less evenly insulinized throughout the day. On this regimen, if the pre-breakfast blood glucose is high, while the 3 a.m. level is low, then the evening dose may need to be split so as to provide short acting insulin before dinner and intermediate acting insulin at bedtime.

Multi-dose insulin regimens: The body normally produces insulin in a basal-bolus manner, i.e., there is a constant basal secretion unrelated to meal intake and superimposed on this there is bolus insulin release in response to each meal. Multi-dose insulin regimens were devised to mimic this physiological pattern of insulin production. Short acting insulin is taken before each major meal (breakfast, lunch and dinner) to provide “bolus insulin” and intermediate acting insulin is administered once or twice a day for “basal insulin.” Usually bolus insulin comprises 60% of the total dose and basal insulin makes up the remaining 40%. With this regimen you have a lot of flexibility. Both the timing as well as the quantity of each meal can be altered as desired by making appropriate alterations in the bolus insulin doses. To take maximum advantage of this regimen, one should learn “carbohydrate counting” and work out carbohydrate:insulin ratio—the number of grams of carbohydrate for which the body needs 1 unit of insulin.

B. Monitoring Glucose Levels

Any person suffering from diabetes will be very familiar with the need to regularly measure blood glucose levels. Blood glucose level is the amount of glucose, or sugar, in the blood. It is also is referred to as “serum glucose level.” Normally, blood glucose levels stay within fairly narrow limits throughout the day (4 to 8 mmol/l), but are often higher after meals and usually lowest in the morning. Unfortunately, when a person has diabetes, their blood glucose level sometimes moves outside these limits. Thus, much of a diabetic's challenge is to When one suffers from diabetes, it is important that glucose level be as near normal as possible. Stable blood glucose significantly reduces the risk of developing late-stage diabetic complications, which start to appear 10 to 15 years after diagnosis with type I diabetes, and often less than 10 years after diagnosis with type II diabetes.

Blood glucose levels can be measured very simply and quickly with a home blood glucose level testing kit, consisting of a measuring device itself and a test strip. To check blood glucose level, a small amount of blood is placed on the test strip, which is then placed into the device. After about 30 seconds, the device displays the blood glucose level. The best way to take a blood sample is by pricking the finger with a lancet. Ideal values are (a) 4 to 7 mmol/l before meals, (b) less than 10 mmol/l one-and-a-half hours after meals; and (c) around 8 mmol/l at bedtime.

People who have type I diabetes should measure their blood glucose level once a day, either in the morning before breakfast or at bedtime. In addition, a 24-hour profile should be performed a couple of times a week (measuring blood glucose levels before each meal and before bed). People who have type II diabetes and are being treated with insulin should also follow the schedule above. People who have type II diabetes and who are being treated with tablets or a special diet should measure their blood glucose levels once or twice a week, either before meals or one-and-a-half hours after a meal. They should also perform a 24-hour profile once or twice a month.

The main advantage for measuring blood glucose levels of insulin-treated diabetics in the morning is that adjusted amounts of insulin can be taken if the blood glucose level is high or low, thereby reducing the risk of developing late-stage diabetic complications. Similarly, the blood glucose level at bedtime should be between 7 and 10 mmol/l. If blood glucose is very low or very high at bedtime, there may be a need to adjust food intake or insulin dose. Blood glucose should also be measured any time the patient does not feel well, or think blood glucose is either too high or too low. People who have type I diabetes with a high level of glucose in their blood (more than 20 mmol/l), in addition to sugar traces in the urine, should check for ketone bodies in their urine, using a urine strip. If ketone bodies are present, it is a warning signal that they either have, or may develop, diabetic acidosis.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Animals. Eight week-old nonobese diabetic (NOD/LtJ) mice from the Jackson Lab were employed for studies of autoimmune diabetes. Ten-week-old lean wild-type (+/+) Zucker Diabetic Fatty (ZDF) rats weighing ˜300 g and fed Teklad 6% fat mouse/rat chow (Teklad, Madison, Wis.) were employed for studies of chemical diabetes. To exclude reduced food intake in hyperleptinemic rodents as the cause of improved metabolic state, in some experiments the food intake of controls was matched to that consumed by hyperleptinemic animals on the previous day. This is referred to as “diet-matching.”

All mice and rats were housed in individual cages with constant temperature, and a standard light/dark cycle (6 a.m. to 6 p.m./6 p.m. to 6 a.m.). All were fed 6% mouse/rat chow diet (Teklad, Madison, Wis.) and had free access to water. All animal protocols were approved by the Institutional Animal Care and Research Advisory Committee of the University of Texas Southwestern Medical Center.

Induction of diabetes by STZ. Normal lean wild-type ZDF (+/+) rats were fasted overnight. A single i.v. injection of 80 mg/kg of body weight of STZ (Sigma, St. Louis, Mo.) in 300 μl of 0.9% saline solution was administered to one group of rats. In another set of experiments 2 doses of 80 mg/kg of body weight were administered with a one-week interval. Blood glucose concentrations were monitored every 3 days after the STZ treatment.

Induction of diabetes by alloxan. After an overnight fast lean wild-type ZDF (+/+) rats received an i.v. injection of alloxan (80 mg/kg of body weight) (Sigma, St. Louis, Mo.) in 150 μl of 0.9% saline. Blood glucose concentrations were monitored after the alloxan treatment.

Adenovirus-induced hyperleptinemia. Beginning at age of 14 weeks old, most NOD mice developed severe hyperglycemia (non-fasting blood glucose above 400 mg/dl) accompanied by marked hypoinsulinemia, ketonuria and cachexia and were obviously near death. These diabetic mice received intravenously a total of 0.3×10¹² plaque-forming units of adenovirus containing either the leptin cDNA (Adv-leptin) or, as a control, the β-galactosidase cDNA (Adv-β-gal). Bodyweight and food intake were monitored daily. Blood glucose concentrations were monitored twice a week.

All STZ- and alloxan-treated rats developed severe diabetes with ketonuria and cachexia. Control rats received Adv-β-gal intravenously and were diet-matched to the leptinized controls. A third group of STZ-diabetic received insulin 5 units (Humulin R, Eli Lilly, Indianapolis, Ind.) i.p. twice with a 30-min interval. Animals were sacrificed and their tissues harvested after blood glucose levels fell to 100 mg/dl or less. In the case of insulin treatment this was 3 hours after treatment; in the case of Adv-leptin treatment it was 3 days.

Tissue collection and preparation. NOD mice were sacrificed under phenobarbital anesthesia at 3 days and at 30 days after treatment and non-fasting blood samples were obtained from the inferior vena cava. Heart, liver, kidney, spleen, muscle and white adipose tissue were rapidly excised and frozen in liquid nitrogen and stored at −80° C.

STZ- and alloxan-diabetic rats were sacrificed under phenobarbital anesthesia 30 days after treatment. Non-fasting blood samples were obtained from the inferior vena cava and tissues of interest were rapidly excised, frozen in liquid nitrogen and stored at −80° C. for protein and RNA extraction.

Plasma measurements. Plasma glucose was measured by PGO glucose kit (Sigma, St Louis, Mo.). Plasma leptin and insulin were assayed using rat leptin ELISA kit and ultra sensitive rat insulin ELISA kit (Crystal Chem. Inc., Downers Grove, Ill., USA). Plasma glucagon was measured by rat glucagon RIA kit (Linco Research, St. Charles, Mo.). Plasma IGF-1 was measured by rat/mouse Insulin-Like Growth Factor (IGF-1) ELISA Kit (Gropep Limited, IDS Inc., Fountain Hills, Ariz.).

Immunocytochemical studies. Fragments of the tail of pancreata were fixed in Bouin's solution and processed for insulin and glucagon immunohistochemistry staining as previously described (Orci et al., 1976). Insulin-positive cells were quantified using Image-J image analysis software and particle analysis macro (Scion, Frederick, Md.). The area of insulin staining in 12 sections of pancreas from 5 animals, relative to total sectional area examined, was quantified by monochromatic thresholding. Pictures were taken with an Axiophot microscope, objective X20.

Immunoblotting analysis. Total protein extracts prepared from tissues of lean +/+ rats were resolved by SDS-PAGE and transferred to a polyvinylidene difluoride membrane (Amersham Biosciences, Piscataway, N.J.). The blotted membrane was blocked in 1×TBS containing 0.1% Tween and 5% nonfat dry milk (TBST-MLK) for 1 h at room temperature with gentle, constant agitation. After incubation with primary antibodies anti-phospho-AKT, anti-phospho-STAT3 (Tyr705) (Cell Signaling Technology, Beverly, Mass.), anti-phospho-CREB (Ser133) (Cell Signaling Technology), anti-phospho-IGF-1 receptor, anti-phospho-MAP kinase (ERK1/2) (Cell Signaling Technology), or anti-tubulin (Sigma, St. Louis, Mo.) in freshly prepared TBST-MLK at 4° C. overnight with agitation, the membrane was washed two times with TBST buffer. This was followed by incubating with secondary anti-rabbit, -mouse, or -sheep horseradish peroxidase-conjugated IgG antibodies in TBST-MLK for 1 h at room temperature with agitation. The membrane was then washed three times with TBST buffer, and the proteins of interest on immunoblots were detected by an enhanced chemiluminescence detection system (Amersham Biosciences). The corresponding bands were quantified using NIH Image J software (version 1.6).

Immunoprecipitation of phospho-IRS-1 and phospho-AKT. Immuno-precipitation was carried out by incubating 2 mg of protein with IRS-1 antibody (Cell Signaling Technology, Beverly, Mass.) overnight, following precipitation of the immunocomplexes with 20 μl protein A-Sepharose, the beads then were washed 5 times in the cell lysis buffer as described above, resolved by SDS-PAGE gel, analyzed by western blotting.

Quantitative real-time RT-PCR. Total RNA was extracted from pancreas and liver by the Trizol isolation method according to the manufacturer's protocol (Life Technologies, Gaithersburg, Md.). All reactions were done in triplicate. The real-time amount of all mRNA was calculated using standard curve method. 18S mRNA was used as the invariant control for all studies. Primer sequences of genes used for quantification of mRNA by real-time PCR appear in Table 1. The primers for preproinsulin mRNA do not distinguish between the 1 and 2 isoforms.

TABLE 1
Primer Sequences Used for Real-Time RT-PCR

| Gene | Forward | Reverse |
|---|---|---|
| Rat AKT-1 | GGCCACTGGCCGCTATT | GCGACTTCATCCTTTGCAATG |
| | (SEQ NO: 1) | (SEQ NO: 2) |
| Rat IGF-1 | ATTCATTTCGCGTTTGGAAAA | CAGACCCAGCACGGAAAGAA |
| | (SEQ NO: 3) | (SEQ NO: 4) |
| Rat PEPCK | GCCTGTGGGAAAACCAACCT | CACCCACACATTCAACTTTCCA |
| | (SEQ NO: 5) | (SEQ NO: 6) |
| Rat PGC-1 | CAGCCAGTACAGCCCTGATGA | TGGTAAGCGCAGCCAAGAG |
| | (SEQ NO: 7) | (SEQ NO: 8) |
| Rat Preproinsulin | TTTGTCAAACAGCACCTTTGTG | GGGTGTGTAGAAGAAACCACGTT |
| | (SEQ NO: 9) | (SEQ NO: 10) |
| Mouse | GGGGAGCGTGGCTTCTTCTA | GGGGACAGAATTCAGTGGCA |
| Preproinsulin | (SEQ NO: 11) | (SEQ NO: 12) |

Statistical analysis. All results are expressed as mean±SEM. The statistical significance of differences in mean values was assessed by Student's t test for two groups.

Monotherapy injection of animals. Eight week-old non-obese diabetic (NOD/LtJ) mice were purchased from the Jackson Lab and housed in individual cages in a temperature-controlled environment with ad libitum access to water and Tekla pelleted 6% fat mouse/rat chow (Teklad, Madison, Wis.) and a standard light/dark cycle (6 am to 6 pm/6 pm. to 6 am). Glucose was measured in conscious animals from a hand-held glucose meter on tail vein blood between 10:00 and 12:00 a.m. at ˜5-day intervals. Animals were sacrificed under sodium pentobarbital anesthesia. Non-fasting blood samples were obtained from the inferior vena cava. All tissues were rapidly excised, frozen in liquid nitrogen, and stored at −70° C. until use. Institutional guidelines for animal care and use were followed. The animal protocol was approved by the Institutional Animal Care and Research Advisory Committee of the University of Texas Southwestern Medical Center at Dallas.

Subcutaneous leptin infusion. Mini-osmotic pumps (Alzet, model 2001) were loaded with recombinant leptin (provided by Amylin Company) at a concentration of 20 mg/ml to delivered at a rate of 1 μl/hr over a 7 day-period. Pumps were implanted subcutaneously between the scapulae under ketamine/xylezine anestesia (0.1 ml/20-30 g body weight) and replaced after 6 days. The untreated control group received phosphate buffered saline (PBS) delivered by the same pump, while the insulin-treated control group received sustained release insulin implants for mice (Linshin Canada, Ltd., Toronto, Canada). Food intake, body weight and blood glucose were monitored daily. Blood samples were collected on days 0, 1, 2, 3, 5, 7, 10 and 12.

Subcutaneous leptin and insulin injections. Diabetic NOD mice received twice daily subcutaneous injections of recombinant leptin plus twice daily subcutaneous injections of the long acting insulin analog levemir (Novo Nordisk) at a dose of 0.01 units. Control mice were treated with insulin monotherapy using levemir 0.01 units or 0.1 units subcutaneously twice daily.

Quantitative Real-Time Polymerase Chain Reaction (QRT-PCR). Total RNA was extracted from tissues by TRIzol isolation method (Life Technologies, Rockville, Md.). All PCRs were done in triplicate, as previously described (Wang et al., 2008). mRNA was calculated by using the standard curve method. 36B4 RNA was used as the invariant control. Primer sequences of genes used for quantification of mRNA by QRT-PCR are shown in Table 1.

Plasma Measurements. Plasma leptin and insulin were measured by using ELISA kits (Crystal Chem, Downers Grove, Ill.). Plasma glucagon was measured using a rat glucagon RIA kit (Linco Research, St. Charles, Mo.). Plasma insulin-like growth factor-1 (IGF-1) was measured by rat/mouse IGF-1 ELISA kit (Gropep Limited, IDS Inc., Fountain Hills, Ariz.). Plasma triglycerides (TG) were measured by using a glycerol phosphate oxidase-Trinder triglyceride kit (Sigma). Plasma-free fatty acids were measured using the Wako NEFA kit (Wako Chemical USA, Richmond, Va.). Plasma cholesterol profiles were performed in the laboratory of Jay Horton, M.D. Glycated hemoglobin Alc was measured by HPLC in the laboratory of Philip Raskin, M.D.

Triacylglycerol (TG) Content of Tissues. Mice were anesthetized with pentobarbital sodium. Tissues were rinsed with PBS (pH 7.4), dissected, and placed in liquid nitrogen immediately. Total lipids from tissues were extracted and dried under N₂ gas. TG content was assayed as previously described (Folch et al., 1957).

### Example 2

### Results

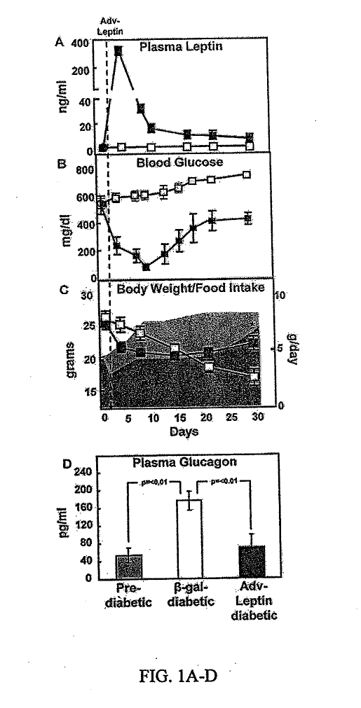

Hyperleptinemia normalizes uncontrolled diabetes in non-obese diabetic (NOD) mice. The NOD mouse is the most commonly employed rodent model of autoimmune or type I diabetes. Seventy-three percent of females (Oge et al., 2007) die with the clinical manifestations of insulin deficiency (Oldstone et al., 1990), unless treated with insulin. Here, the inventor studied 20 female NOD mice, 15 of which developed diabetes between 12-20 weeks of age. Six of the diabetic mice were either untreated or received adenovirus containing the cDNA of β-galactosidase (Adv-β-gal) as an irrelevant control. There were no clinical differences between untreated and Adv-β-gal-treated mice. Nine mice were treated with adenovirus containing the leptin cDNA (Adv-leptin), inducing marked hyperleptinemia averaging 319±76 ng/ml 3 days after the injection (FIG. 1A). Leptin levels decreased rapidly thereafter, measuring 16±4 ng/ml by the 9^{th} post-injection day. For at least 30 days after treatment they remained above 1 ng/ml, the mean leptin level in untreated diabetic animals.

The non-fasting glucose levels of the controls averaged 534±199 mg/dl before Adv-leptin treatment (FIG. 1B). Urine was strongly positive for glucose and ketones (data not shown). Plasma insulin levels in the fed state were all below 0.1 ng/ml by 18 weeks of age, compared to 1.4±0.1 ng/ml in normal non-diabetic controls. Glucose levels fell to normal in every Adv-leptin-treated mouse from over 534±199 mg/dl to 77±67 mg/dl at 9 days after infection (FIG. 1B). Subsequently, however, it gradually increased and by 22 days after Adv-leptin injection it had risen to 410±146 mg/dl. At this point it seemed to reach a plateau, never approaching the ˜600 mg/dl range of untreated animals during 4 weeks of observation. Moreover, there was no weight loss or apparent deterioration in their health. In addition to lack of measurable plasma insulin, pancreatic preproinsulin mRNA was undetectable (CT ˜34) in both the leptinized diabetic mice and in the untreated controls, using primer sequences that do not differentiate between preproinsulins 1 and 2, while preproglucagon mRNA was increased (CT ˜23). Thus, the metabolic improvement in the former group of NOD mice appears to have occurred independently of insulin.

Hyperleptinemia reduced food intake to 51% of the control diabetic NOD mice given Adv-β-gal (data not shown). Despite their hyperphagia, body weight of the control mice declined, while in hyperleptinemic mice the weight loss halted at ˜7 days after Adv-leptin injection and body weight rose slightly thereafter (FIG. 1C). Pair-feeding of untreated controls to the hyperleptinemic mice proved to be lethal within 4 days of diet restriction and was discontinued. Remarkably, the leptinized group appeared to thrive and was without abnormalities in appearance or behavior.

Suppression of diabetic hyperglucagonemia in NOD mice by hyperleptinemia. Hyperglucagonemia is present in insulin deficiency states (Muller et al., 1971) and is essential for the hepatic overproduction of glucose and ketones of uncontrolled diabetes (Dobbs et al., 1975). To determine if suppression of hyperglucagonemia by leptin accounted for the reversal of the extreme catabolic state, the inventor compared glucagon levels in untreated and treated diabetic mice. Plasma glucagon measured 175±21 pg/ml in untreated mice, significantly higher than levels in prediabetic NOD mice (53±17 pg/ml) (p<0.01) (FIG. 1D). Thirty days after Adv-leptin treatment glucagon levels averaged 69±28 pg/m, significantly below the Adv-β-gal-treated mice (p<0.01). The latter value was not significantly different from plasma glucagon levels in the prediabetic mice. Thus leptin-mediated suppression of diabetic hyperglucagonemia may contribute to the reversal of the diabetic state.

Hyperleptinemia normalizes the uncontrolled diabetes of STZ and alloxan diabetic rats. To determine if hyperleptinemia would be as effective in other forms of diabetes in another species, the inventor studied its effects in rats with chemically induced f3-cell destruction. Six normal, lean wild-type Zucker Diabetic rats received 80 mg/kg of STZ, their maximal sublethal dose, and 11 rats received 100 mg/kg of alloxan. All untreated animals died in less than 3 months with severe hyperglycemia and ketoacidosis.

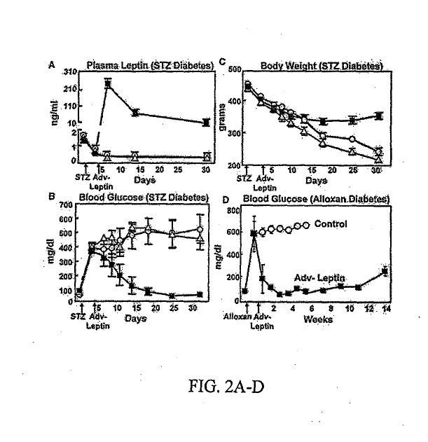

A single intravenous injection of 10¹² plaque-forming units of Adv-leptin induced hyperleptinemia of ˜300 ng/ml at 3 days, after which levels declined slowly to 20 ng/ml by the 30^{th} day (FIG. 2A). Glucose levels averaging 400±96 mg/dl were restored to normal within 18 days in all 6 STZ-diabetic rats and normoglycemia persisted throughout a 30-day observation period (FIG. 2B). During this period the progressive weight loss of uncontrolled diabetes was halted and, remarkably, body weight increased despite the hyperleptinemia (FIG. 2C). Treatment with Adv-β-gal had no effect on the diabetes. The effects of hyperleptinemia on other relevant clinical and laboratory manifestations of uncontrolled diabetes are recorded in Table 2. Similar normalization of hyperglycemia was observed in all 11 alloxan-diabetic rats (FIG. 2D), and the improvement persisted for ˜80 days without any other therapy.

TABLE 2
Metabolic Profiles of Untreated STZ and Adv-leptin Treated STZ Rats and
Non-diabetic (nd) Controls 30 Days after Time of Treatment (n = 6)

| | | | | p value | p value |
|---|---|---|---|---|---|
| | | | | (untreated | (adv- |
| | | | | vs | leptin |
| Measure- | | | | Adv- | vs |
| ment | Untreated | Adv-leptin | nd | leptin) | lean) |
| Blood | 678 ± 17 | 99 ± 57.4 | 74 ± 6 | 0.001 | 0.18 |
| glucose, | | | | | |
| mg/dl | | | | | |
| Urine | 1000-2000 | negative | negative | | |
| glucose, | | | | | |
| mg/dl | | | | | |
| Insulin, | 0 | 0 | 1.4 ± 0.1 | | |
| ng/ml | | | | | |
| Leptin, | 0.02 ± 0.02 | 20.4 ± 5.9 | 1.7 ± 0.5 | 0.006 | 0.008 |
| ng/ml | | | | | |
| TAG, | 1062 ± 236 | 9 ± 2 | 50 ± 12 | 0.011 | 0.02 |
| mg/dl | | | | | |
| FFA, | 2.2 ± 1.2 | 0.19 ± 0.1 | 0.3 ± 0.2 | 0.04 | 0.42 |
| mEq/ml | | | | | |
| Liver | 1.1 ± 0.5 | 4.7 ± 0.8 | 6.8 ± 0.8 | 0.003 | 0.02 |
| TAG, | | | | | |
| mg/g | | | | | |
| Muscle | 0.3 ± 0.1 | 1.6 ± 0.3 | 3.4 ± 1.1 | 0.02 | 0.04 |
| TAG, | | | | | |
| mg/g | | | | | |

In a separate longer-term study encompassing 174 days, hyperglycemia slowly reappeared but reached a plateau well below the pretreatment levels and the animals remained in apparent good health (FIG. 6).

Thus, as in the NOD mice, hyperleptinemia reversed the metabolic and clinical manifestations of chemically induced β-cell destruction in the absence of any insulin.

Potentiation of residual insulin as the mechanism of hyperleptinemic action. Although potentiation of residual insulin was excluded as the mechanism of hyperleptinemic reversal of NOD diabetes, it seemed important to confirm this in chemically-induced diabetes as well. The nonfasting plasma insulin levels in the streptozotocin-diabetic rats were very low after the Adv-leptin treatment of the diabetic rats (0.2±0.03 ng/ml before and 0.18±0.07 ng/ml after), versus 1.4±0.3 ng/ml level of nonfasting plasma insulin in normal rats. Nevertheless, they were higher than the “zero” reading on the standard curve.

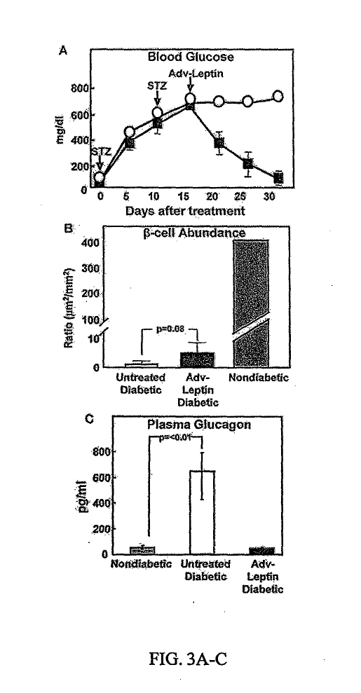

Therefore, to rule out the possibility that hyperleptinemia had potentiated these miniscule insulin levels, the inventor administered the 80 mg/kg dose of streptozotocin twice (2XSTZ) to 9 normal rats in an effort to achieve more complete β-cell destruction. These rats exhibited mean blood glucose levels of 674±18 mg/dl without treatment, and their plasma insulin levels below the detection levels of the assay. The induction of hyperleptinemia in both the 2XSTZ diabetic rats elicited the same progressive decline in glucose levels to normal and complete clinical improvement within 14 to 20 days (FIG. 3A). Immunostaining of the pancreata of Adv-leptin-treated rats still revealed 1 or 2 insulin-positive cells per 10-15 islets (FIG. 3B), which, although not statistically different, was more than in the untreated controls (p=0.08). However, once again preproinsulin mRNA could not be detected in the pancreas by quantitative RT-PCR (CT>34), although preproglucagon mRNA was readily detected (CT ˜23). This suggests that these rats were incapable of insulin biosynthesis, and raises the possibility the very rare insulin-positive cells in the pancreas represent insulin granules trapped in badly damaged nonfunctional β-cells undergoing apoptosis and/or macrophages that had engulfed insulin granules.

Finally, the possibility of extrapancreatic insulin production, reported in liver of insulin-deficient rodents (Kojima et al., 2004; Sapir et al., 2005), was also examined. The inventor was unable to identify in liver any preproinsulin mRNA by quantitative RT-PCR (CT>35), and therefore conclude that the antidiabetic effect of hyperleptinemia in chemically induced β-cell destruction is unlikely to be mediated by potentiation of endogenous pancreatic or extrapancreatic insulin.

Suppression of diabetic hyperglucagonemia by hyperleptinemia in STZ-induced diabetic rats. To determine if suppression of hyperglucagonemia by hyperleptinemia contributed to the antidiabetic effect in STZ-diabetic rats, the inventor measured plasma glucagon before and 30 days after injection of the Adv-leptin. Glucagon levels before the STZ induction of diabetes averaged 63±35 pg/ml. Thirty days after the onset of untreated STZ diabetes they measured 649±205 pg/ml. Thirty days after treatment with Adv-leptin plasma glucagon had declined to 46±6 pg/ml (p<0.0.1) (FIG. 3C). Thus, as in NOD mice, glucagon suppression may have contributed to the reversal of the chemically-induced uncontrolled diabetic state in rats.

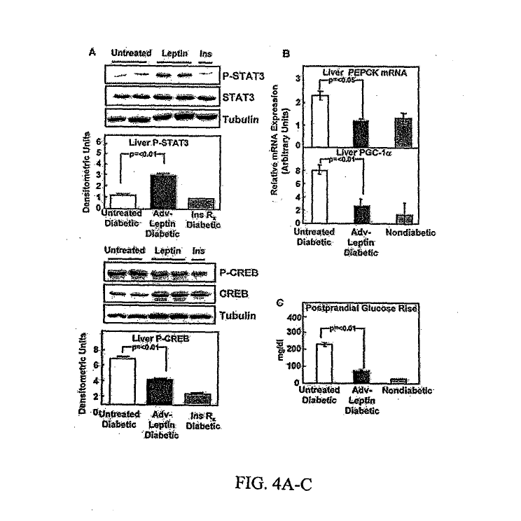

Inhibition of hepatic glucagon action by hyperleptinemia in STZ-induced diabetic rats. Insulin treatment reverses the excess glycogenolysis, gluconeogenesis and ketogenesis of insulin deficiency, not only by suppressing the hypersecretion of glucagon, but also by direct action on the liver to inhibit the hepatic effects of any unsuppressed plasma glucagon. To determine if the hyperleptinemia had acted directly on the liver, the inventor measured hepatic phospho-STAT3, an index of leptin action mediated via the hepatic leptin receptor. P-STAT3 was significantly increased (p<0.01) (FIG. 4A). This provides evidence for autocrine activity of the liver-derived hyperleptinemia on hepatocytes infected with Adv-leptin.

Glucagon action on the liver increases the phosphorylation of cyclic AMP response element binding protein (P-CREB) (Dalle et al., 2004). P-CREB was elevated in the livers of untreated STZ-induced diabetic rats with hyperglucagonemia. Both insulin treatment and treatment with Adv-leptin significantly reduced P-CREB (p<0.01). At 30 days after treatment, P-CREB in the hyperleptinemic rats was significantly lower than in untreated rats (p<0.01), although not as low as 3 hrs after insulin treatment (FIG. 4A).

Based on these findings, one would predict a reversal of the increased hepatic gluconeogenesis of uncontrolled diabetes. Such a reversal would be reflected by decreased expression of phoshoenolpyruvate carboxykinase (PEPCK), a key gluconeogenic enzyme that is stimulated by glucagon (Christ et al., 1988), which is elevated in uncontrolled diabetes. In untreated STZ-diabetic rats PEPCK mRNA was 48% above normal and declined by 43% at 30 days after the Adv-leptin treatment (FIG. 4B). In addition, Adv-leptin treatment reduced peroxisome proliferator-activated receptor-γ-coactivator-1α (PGC-1α) mRNA, which is also implicated in activation of gluconeogenesis (Matsumoto et al., 2007) (FIG. 4B). The reduction by hyperleptinemia of these gluconeogenic proteins may well contribute importantly to the reversal of the catabolic state.

The suppression of glucagon and its activity by hyperleptinemia would also be expected to inhibit ketogenesis (Exton et al., 1969), although leptin itself causes non-ketotic fatty acid oxidation of lipids (Lee et al., 2002). In untreated insulin-deficient rats hepatic TAG content had declined from a normal value of 6.8±0.8 mg/g of tissue weight to 1±0.5 mg/g. Thirty days after the induction of hyperleptinemia it measured 4.7±0.8 mg/g of liver. Remarkably, plasma triacylglycerol (TAG), which averaged over 1000 mg/dl in untreated diabetic rats, was less than 9 mg/dl in hyperleptinemic rats (Table 2), suggesting that a profound reduction in the secretion of very low density lipoproteins (VLDL) coincided with the increase in hepatic TAG content. Plasma free fatty acids (FFA) in untreated STZ rats averaged 2.2±1.2 mEq/L, almost 8 times the value in lean nondiabetic rats (p<0.004); one month after Adv-leptin treatment they measured 0.19±0.06 mEq/L (Table 2). This is most probably reflected the loss of adipocyte fat due to the lipolytic consequences of insulin deficiency coupled with the lipo-oxidative action of the hyperleptinemia (Orci et al., 2004).

Insulinomimetic activity by hyperleptinemia in STZ-induced diabetic rats. To determine if hyperleptinemia mimics the extra-hepatic actions of insulin, the inventor compared fasting and nonfasting glucose levels in normal, untreated STZ-diabetic, and Adv-leptin-treated STZ-diabetic rats (FIG. 4C). In normal rats, the fasting and postprandial glucose levels differed by only 26±1.2 mg/dl. In untreated diabetic rats, they differed by 227±11 mg/dl, whereas in the leptinized group the difference was 74±6 mg/dl at 30 days after treatment. Thus, it appears that leptin action reduces postprandial hyperglycemia in insulin-deficient rats.

Activation of the insulin signal transduction pathway by hyperleptinemia. To determine if the insulin-like actions of hyperleptinemia involved the activation of elements of the insulin signal transduction pathway, the inventor compared the phosphorylation of insulin receptor substrate (IRS)-1, phosphotidylinositol-3-kinase (PI3K), protein kinase B (Akt)-1 and extracellular signal-regulated kinase (ERK) in the livers and skeletal muscles of untreated insulin-deficient rats, insulin-treated and Adv-leptin-treated diabetic rats. Unlike insulin, leptin had no significant effect on any of these 4 insulin targets in liver, despite an almost 3-fold increase in hepatic phosho-STAT-3 (p<0.0001) (data not shown). Since in the liver of normal rodents leptin induces a 6.8-fold activation of mitogen-activated protein kinase (MAPK) (Kim et al., 2004), the inventor suspected that this difference reflects leptin-mediated potentiation of insulin's hepatic action, which was lacking in our insulin-deficient rodents.

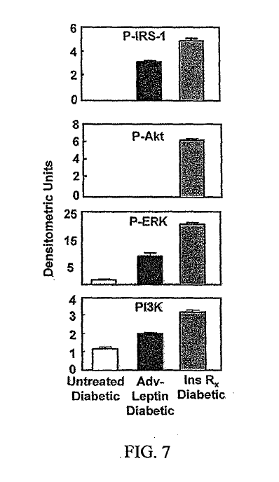

However, in skeletal muscle the effects of hyperleptinemia were more insulin-like. P-IRS-1, which was undetectable in untreated diabetic rats, was increased in the hyperleptinemic rats. At 3 days after Adv-leptin injection, it measured over 60% of the value observed 3 hours after insulin injection (FIG. 7). PI3K in skeletal muscle of leptinized rats was also significantly greater than in untreated diabetics, measuring over 60% of the value noted 3 hours after insulin administration (p<0.001). P-ERK was increased 9-fold above the level in muscle of untreated diabetic rats to 44% of the insulin-induced increment. On the other hand, there was no increase in P-Akt in either liver or muscle of rats 30 days after induction of hyperleptinemia. These results suggest that, although in insulin-deficient rodents the hepatic effects of hyperleptinemia do not involve the insulin-signaling pathway, those in skeletal muscle may be mediated by certain components of the pathway.

Leptin induction/potentiation of insulinomimetic hormones. The activation by hyperleptinemia of insulin signaling molecules in skeletal muscle, but not in liver, raised the possibility of potentiation of an insulinomimetic hormone. Fibroblast growth factor (FGF)-21 is reported to have insulinomimetic properties that could play a regulating role in metabolism (Kharitonenkov et. al., 2008). However, FGF21 mRNA in livers of STZ rats was not upregulated by hyperleptinemia (data not shown).

A role for insulin-like growth factor (IGF)-1, which can activate ERK (Choi et al., 2008), was also considered. A comparison of 3 untreated and 4 Adv-leptin treated STZ-diabetic rats revealed plasma IGF-1 to be significantly increased in the hyperleptinemic rats 30 days after treatment (p<0.01 (FIG. 5A). Hepatic IGF-1 mRNA was upregulated (p<0.01) in the Adv-leptin-treated rats at this time (FIG. 5B). To determine if the plasma IGF-1 elevation was acting on target tissues, the inventor measured phosphorylation of IGF-1 receptor in liver and muscle. At 3 days post-treatment, but not at 30 days, they found a significant increase in P-IGF1-R in skeletal muscle of hyperleptinemic rats (FIG. 5C), the tissue in which components of the insulin signaling pathway had been activated. The inventor found no such increase in liver, in which they had not been activated. These findings are consistent with IGF-1 mediation of the insulin-like action of hyperleptinemia in skeletal muscle. They may also be relevant to the impressive increase in body weight and linear growth observed in Adv-leptin-treated diabetic animals (FIG. 5D).

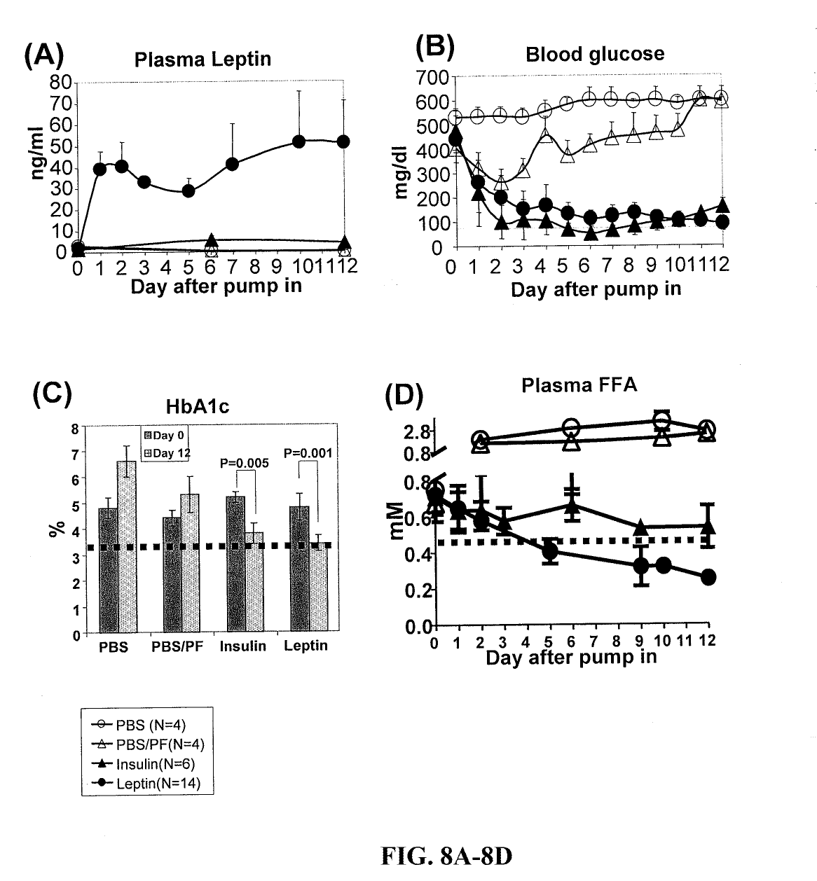

Injection of leptin reduces glucose levels in NOD mice. To determine if leptin monotherapy is effective in type I diabetes, fifteen diabetic NOD mice with hyperglycemia ranging from 220 to 572 mg/dl underwent implantation of an Alzet Osmotic pump containing 3.3 mg of leptin so as to deliver 20 μg of leptin/h for 12 days. They were compared with diabetic littermates treated with insulin by subcuteous pellet. Untreated controls received PBS infusion by Alzet pump. Mean plasma leptin levels ranged between 20 and 50 ng/ml during the period of leptin infusion (FIG. 8A). Plasma glucose levels declined in all fifteen leptin-treated animals, averaging 88±28 mg/dl after 12 days, compared to 160±32 mg/dl on the insulin pellet (FIG. 8B; Table 3). Ketonuria, which ranged from 40-160 mg/dl in severely hyperglycemic PBS-treated control mice, disappeared with both leptin and insulin treatment (Table 3). Like insulin treatment, leptin infusion lowered hemoglobin Alc to 3.4±0.3%, similar to the level in non-diabetic littermates (FIG. 8C; Table 3). Plasma FFA were also dramatically lowered within 24 h by leptin to 0.25±0.04 mM after 12 days of treatment (p<0.03), compared to 0.54±0.1 mM in the insulin group (P=0.08) and 1.9±0.4 mM in PBS-treated controls (FIG. 8D; Table 3). The time required to restore normoglycemia with leptin therapy varied with the severity and duration of the disease, ranging from 1 day in the mice with the least severe mice, to 7-9 days in mice with more severe diabetes of longer duration (data not shown).

Leptin treatment profoundly reduced food intake from 10±1.5 g/d in untreated hyperphagic diabetic mice to 2.8±0.8 g/d, not significally different from the 3.3 g/d intake of normal non-diabetic mice (Table 3). The leptin-treated mice lost 2.5 g of body weight and 77% of body fat during the 12 days, whereas the PBS-treated mice on ad lib feeding lost 2±1.7 g of body weight in 12 days, 50% of which was body fat. When pairfed to the leptinized group, the PBS-treated mice lost 4.4±0.5 g. Insulin-treated mice gained 1.7±1.2 g and their body fat rose 65% (Table 3). Body length in leptin-treated mice measured 8.7±0.5 cm versus 8.4± with insulin therapy (N.S.) and 7.95±0.05 cm in PBS-treated controls (p<0.035). These findings suggest that leptin-induced weight loss was at the expense of body fat rather than lean body mass. Furthermore, liver glycogen increased from 8.5± mg/g to 20±5 mg/g, not different from insulin therapy, which suggests that catabolic actions of leptin are confined to lipids, and that proteins and carbohydrates are exempt.

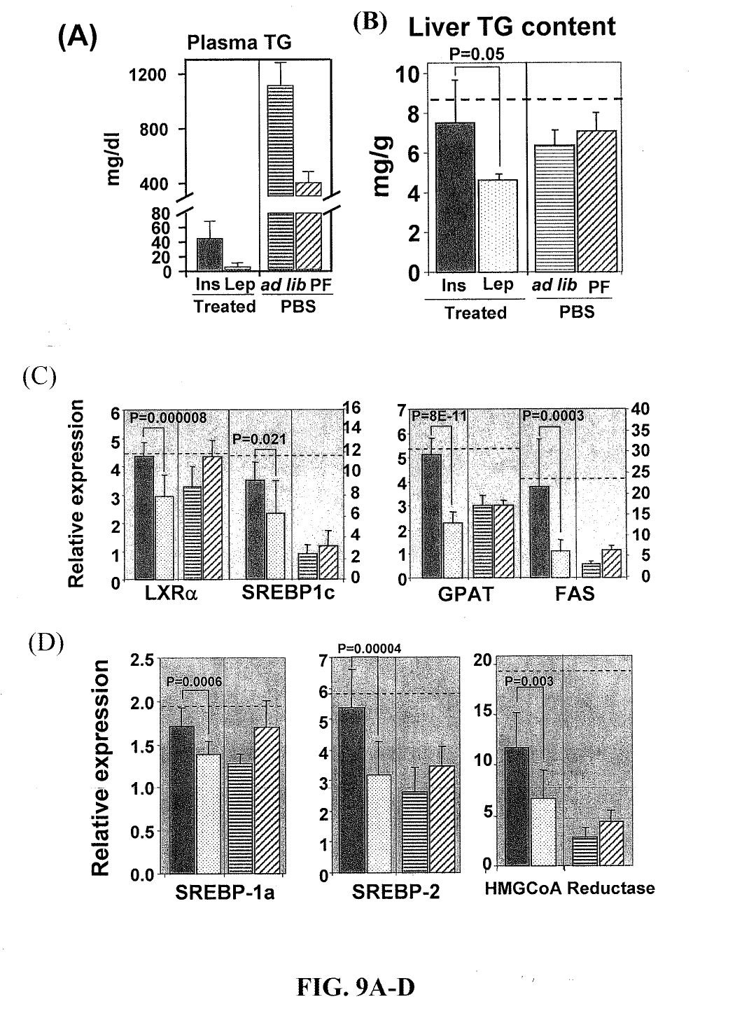

Indeed, the most striking differences between leptin and insulin therapies were in lipid metabolism. In addition to the lowering of FFA, other lipid abnormalities associated with insulin-treated T1DM were corrected by leptin therapy. Plasma triacylglycerol (TG) averaged 1118±165 mg/dl in PBS-treated diabetic mice, and 406±79 mg/dl in the pairfed group, measured 7±5 mg/dl after 12 days of leptin therapy (FIG. 9A; Table 3), compared to 48±23 mg/dl in the insulin-treated group and 31±7 mg/dl in non-diabetic mice. Liver TG averaged 6.4±0.9 mg/g wet weight in PBS-treated diabetic mice, compared to 4.6±0.5 mg/g after 12 d of leptin treatment, 7.5±2 mg/g after insulin and 8.7±1 mg/g. in non-diabetic livers (FIG. 9B; Table 3).

TABLE 3
Metabolic profiles of NOD mice treated with PBS, PBS/pair-fed, recombinant leptin pump,
and insulin pellet, and nondiabetic controls.

| | PBS pump | | PBS/PF | | Leptin |
|---|---|---|---|---|---|
| | (N = 4) | | (N = 4) | | (N = 14) |
| Measurement | Day 0 | Day 12 | Day 0 | Day 12 | Day 0 |
| Body weight, g | 23.9 ± 0.65 | 21.93 ± 1.4* | 23.0 ± 0.95 | 18.63 ± 1.24 | 24.6 ± 2.0 |
| Food intake, g/day | | 10.1 ± 1.6* | | 2.8-3.0 | |
| Body fat, (%) | 11.02 ± 4.72 | 4.71 ± 1.62* | 10.29 ± 1.72 | 3.82 ± 1.07 | 13.49 ± 2.90 |
| Body temperature, ° C. | 37.48 ± 0.76 | 38.15 ± 0.50 | 37.67 ± 0.13 | 37.88 ± 0.33 | 37.24 ± 0.33 |
| Urine glucose, mg/dl | 250-500 | 1000-2000* | 250-500 | 1000-2000 | 250-500 |
| Urine ketone, mg/dl | Negative | 40-160 | Negative | 15-80 | Negative |
| Blood glucose, mg/dl | 531 ± 27 | >600* | 403 ± 98 | 591 ± 14 | 443 ± 97 |
| Hemoglobin A1c, % | 4.8 ± 0.4 | 6.6 ± 0.6* | 4.4 ± 0.3 | 5.3 ± 0.7 | 4.8 ± 0.5 |
| Leptin, ng/ml | 2.38 ± 0.99 | 0.25 ± 0.04* | 2.98 ± 1.38 | 0.27 ± 0.01 | 3.9 ± 2.1 |
| Insulin, ng/ml | 1.54 ± 0.76 | 0.04 ± 0.02 | 1.01 ± 0.11 | 0.05 ± 0.03 | 2.00 ± 1.18 |
| Glucagon, pg/ml | | 391.8 ± 41.6* | | 463.4 ± 111.6 | |
| TG, mg/dl | 63.5 ± 20.5 | 1118.5 ± 165.0* | 51.4 ± 6.4 | 406.7 ± 79.4 | 42.8 ± 7.7 |
| FFA, mM | 0.75 ± 0.14 | 1.88 ± 0.42* | 0.68 ± 0.05 | 1.62 ± 0.05 | 0.72 ± 0.09 |
| Liver TG, mg/g | | 6.36 ± 0.85* | | 7.07 ± 0.63 | |
| Liver glycogen, mg/g | | 7.29 ± 1.68* | | 8.47 ± 6.18 | |

| | Leptin | Insulin | | |
|---|---|---|---|---|
| | (N = 14) | (N = 6) | | Nondiabetic |
| Measurement | Day 12 | Day 0 | Day 12 | (N = 5) |
| Body weight, g | 22.13 ± 1.6^{†} | 24.24 ± 0.91 | 25.94 ± 0.63 | 22.88 ± 0.88 |
| Food intake, g/day | 2.8 ± 0.8^{†} | | 5.9 ± 0.73 | 3.32 ± 0.71 |
| Body fat, (%) | 3.23 ± 1.35^{†} | 8.0 ± 0.99 | 13.2 ± 1.22 | 12.30 ± 2.11 |
| Body temperature, ° C. | 37.84 ± 0.40^{†} | 37.32 ± 0.38 | 37.06 ± 0.61 | 37.64 ± 0.44 |
| Urine glucose, mg/dl | Negative | 250-500 | Negative | Negative |
| Urine ketone, mg/dl | Negative | Negative | Negative | Negative |
| Blood glucose, mg/dl | 88 ± 28^{†} | 477 ± 91 | 160 ± 32 | 102 ± 12 |
| Hemoglobin A1c, % | 3.4 ± 0.3^{†} | 5.2 ± 0.2 | 3.8 ± 0.4 | 3.3 ± 0.1 |
| Leptin, ng/ml | 35.6 ± 11.1^{†} | 1.37 ± 0.88 | 4.19 ± 0.88 | 2.43 ± 0.94 |
| Insulin, ng/ml | 0.06 ± 0.05^{†} | 1.51 ± 0.79 | 11.45 ± 0.47 | 1.32 ± 0.42 |
| Glucagon, pg/ml | 78.5 ± 40.1 | | 53.6 ± 17.6 | 76.5 ± 11.6 |
| TG, mg/dl | 6.7 ± 5.0^{†} | 77.5 ± 30.5 | 47.5 ± 23.1 | 30.9 ± 7.1 |
| FFA, mM | 0.25 ± 0.04^{†} | 0.71 ± 0.14 | 0.54 ± 0.12 | 0.49 ± 0.10 |
| Liver TG, mg/g | 4.61 ± 0.47^{†} | | 7.50 ± 2.14 | 8.65 ± 1.41 |
| Liver glycogen, mg/g | 20.77 ± 5.18 | | 19.33 ± 5.04 | 12.80 ± 4.49 |

*Significant difference between PBS and leptin groups at day 12.
^{†}Significant difference between leptin and insulin groups at day 12.

TABLE 4
Quantitative PCR analysis of mRNAs in the liver tissue of NOD mice treated
with PBS, PBS/pair-fed, recombinant leptin pump, and insulin pellet, and nondiabetic
controls 12 days after time of treatment by using 36B4 as the invariant control.

| | | | | | | | |
|---|---|---|---|---|---|---|---|
| | | P value | | | | P value | Non- |
| | PBS | (Lep vs | PBS/PF | Leptin | Insulin | (Lep vs | diabetic |
| Genes | (N = 4) | PBS) | (N = 4) | (N = 6) | (N = 6) | Ins) | (N = 4) |

(A) FA metabolism

| | | | | | | | |
|---|---|---|---|---|---|---|---|
| LXRα | 3.3 ± 0.7 | | 4.3 ± 0.6 | 2.9 ± 0.8 | 4.4 ± 0.5 | ††† | 4.4 ± 0.4 |
| SREBP1c | 2.4 ± 0.8 | ** | 3.2 ± 1.4 | 6.2 ± 3.3 | 9.3 ± 1.6 | † | 12.2 ± 3.8 |
| FAS | 3.3 ± 0.5 | ** | 6.7 ± 1.0 | 6.4 ± 3.8 | 21.8 ± 12.2 | ††† | 24.1 ± 6.6 |
| GPAT | 3.0 ± 0.4 | ** | 3.0 ± 0.2 | 2.3 ± 0.5 | 5.1 ± 0.7 | ††† | 5.4 ± 0.4 |
| SCD-1 | 16.1 ± 20.5 | *** | 8.4 ± 2.4 | 394 ± 136 | 313 ± 205 | | 484 ± 141 |
| PPARα | 3.3 ± 1.3 | *** | 3.6 ± 0.6 | 6.1 ± 1.5 | 7.4 ± 2.7 | †† | 6.8 ± 0.5 |
| Foxo1 | 1.8 ± 0.2 | *** | 1.6 ± 0.4 | 1.3 ± 0.2 | 1.5 ± 0.2 | | 1.8 ± 0.3 |
| PGC1α | 9.5 ± 4.2 | *** | 6.0 ± 1.8 | 3.0 ± 1.0 | 4.5 ± 2.0 | † | 3.6 ± 1.0 |
| CD36 | 16.9 ± 5.3 | *** | 17.7 ± 5.0 | 3.1 ± 1.3 | 3.6 ± 2.2 | | 2.1 ± 0.4 |
| SPT1 | 5.2 ± 0.6 | * | 5.7 ± 0.8 | 4.3 ± 1.1 | 5.9 ± 0.9 | ††† | 6.3 ± 0.6 |
| SIRT1 | 2.6 ± 0.3 | * | 2.2 ± 0.4 | 3.2 ± 0.7 | 3.8 ± 0.6 | † | 3.2 ± 0.6 |

(B) Gluconeogenesis

| | | | | | | |
|---|---|---|---|---|---|---|
| PEPCK | 5.5 ± 0.5 | *** | 4.7 ± 0.2 | 3.4 ± 0.6 | 3.2 ± 0.9 | 2.7 ± 0.4 |

(C) Cholesterol metabolic

| | | | | | | | |
|---|---|---|---|---|---|---|---|
| SREBP1a | 1.3 ± 0.1 | | 1.7 ± 0.3 | 1.4 ± 0.2 | 1.7 ± 0.2 | ††† | 1.9 ± 0.1 |
| SREBP2 | 2.6 ± 0.8 | | 3.5 ± 0.7 | 3.2 ± 1.1 | 5.4 ± 1.2 | ††† | 5.9 ± 0.5 |
| HMG-CoA R | 2.9 ± 0.9 | ** | 4.3 ± 1.3 | 6.6 ± 3.7 | 11.8 ± 3.4 | †† | 19.2 ± 9.5 |

P values > 0.05 (Insulin VS leptin): MCD, CPT1, IGF1, IGFBP2, BCl2, BAX, Foxc2, PPARα, IRS2, ACCα, ACCβ, ACO, and UCP2.
Leptin vs PBS: * p values < 0.05; ** p value < 0.01; *** p value < 0.001.
Leptin vs Insulin: † p value < 0.05; †† p value < 0.01; ††† p value < 0.001

To determine the mechanism of the anti-lipogenic effect, the inventor compared the expression of the lipogenic transcription factor, sterol regulatory element binding protein (SREBP)-1c, and several of its lipogenic target enzymes (FIG. 9C; Table 4A). The expression of both SREBP-1c and of liver X receptor-α (LXRα), which responds to insulin by activating the SREBP-1c promoter (Chen et al., 2004), was significantly lower in leptin-treated mice than in insulin-treated or in non-diabetic mice (p<0.02; p<0.0001). Expression of two lipogenic enzymes, fatty acyl CoA synthetase (FAS) and glycerophosphate acyl transferase (GPAT), was also far below the levels in nondiabetic or insulin-treated diabetic livers (p<0.0003) (FIG. 9C, Table 2A). IRS-2 mRNA, which is reduced when lipogenesis and resistance to the antigluconeogenic action of insulin are increased (Shimomura et al., 2000), was almost twice that of insulin-treated mice (N.S.) (Table 2A). There were two unexpected results: first, the mRNA of stearoyl CoA desaturase 1 (SCD1) was ˜47-fold higher in both leptin- and insulin-treated mice than in PBS-treated diabetic controls, and second, CD36, the fatty acid transporter, declined to near-normal from elevated levels in the PBS-treated group (Table 4A).

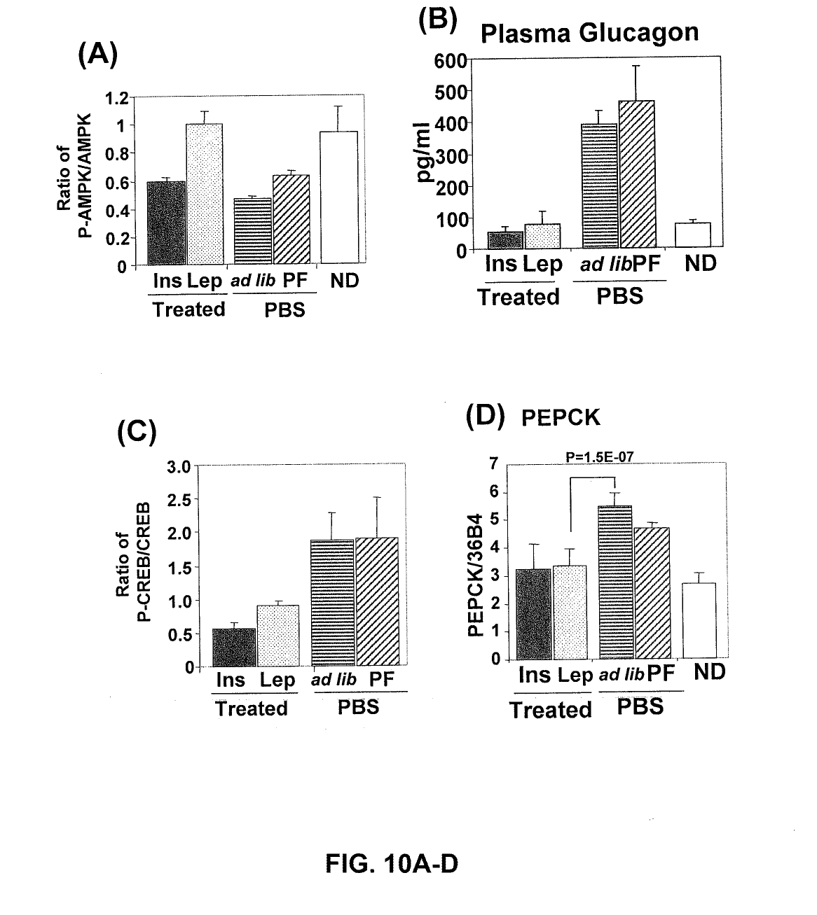

In addition, there was indirect evidence that increased fatty acid oxidation contributed to the lipid-lowering action of leptin. Liver PPARα expression, which was low in PBS-treated diabetic mice, was increased to normal by treatment with both leptin (p<0.0002) and insulin (p<0.007) (Table 4A). Phosphorylated AMP-activated protein kinase (AMPK), a master regulator of β-oxidation of fatty acids (Hardie et al., 1998), was significantly higher in leptin-treated livers than in insulin-treated and untreated mice, confirming earlier work (Minokoshi et al., 2002) (FIG. 10A).

Coronary artery disease (CAD), a common event in longstanding T1DM (Orchard et al., 2003), is generally attributed to the hyperglycemia of diabetes, rather than to the hyperinsulinemia required to treat it. However, because hyperinsulinemia is a risk factor for CAD, the inventor compared in mice treated with leptin or insulin the hepatic expression of two cholesterologenic transcription factors, SREBP1a, and SREBP2 (Briggs et al., 1993), and the rate-limiting enzyme of cholesterol synthesis, HMG CoA reductase. All three were significantly lower with leptin than with insulin treatment (p<0.0006; p<0.0001; p<0.003) (FIG. 9D; Table 4C).

The inventor has previously shown that the hepatic overproduction of glucose and ketones and that these catabolic manifestations of insulin deficiency cannot occur in the absence of hyperglucagonemia (Dobbs et al., 1975). To determine if the anti-diabetic effects of leptin might be mediated by glucagon suppression, as suggested by Tuduri et al. (2009), the inventor compared plasma glucagon in the 4 groups of diabetic mice. It averaged 392±42 and 463±12 pg/ml, respectively, in ad lib fed and pair-fed PBS-treated diabetic controls, and was suppressed to 79±40 pg/ml by leptin therapy and to 54±18 pg/ml by insulin treatment (FIG. 10B, Table 3). Glucagon suppression was associated with a reduction in phosphorylated cAMP response element binding protein (CREB) in the liver, consistent with less glucagon action (FIG. 10C). The mRNA of phosphoenolpyruvate carboxykinase, a prime gluconeogenic target of glucagon, was also reduced by leptin as well as by insulin (FIG. 10D). An increase in hepatic P-STAT3 in the leptin-treated, but not insulin-treated mice, suggests that leptin was acting directly on liver (data not shown).

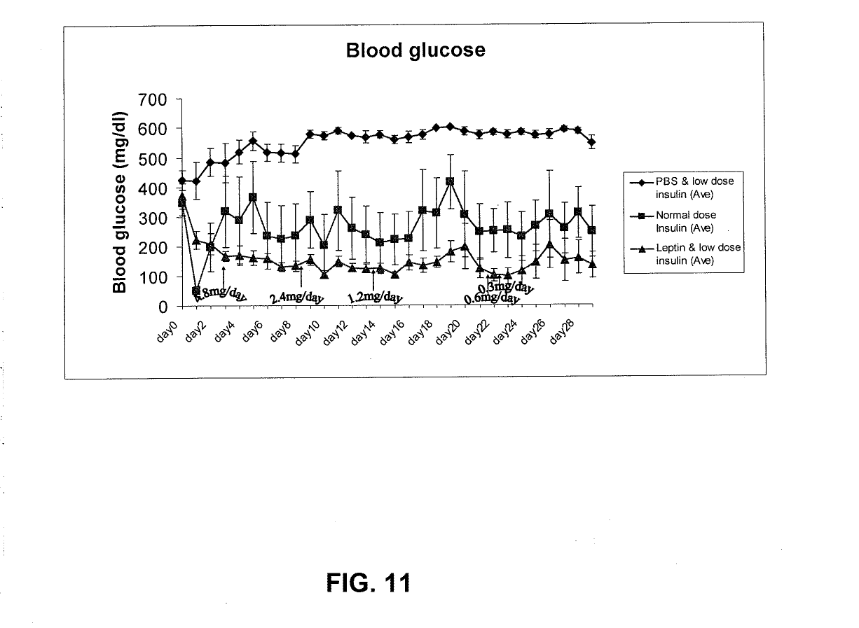

The inventor next compared the effects of leptin alone or supplemented with insulin at 0.02 U/d, or 10% of the optimal insulin dose of 0.2 U/d subcutaneously twice daily. FIG. 11 compares the glycemia of diabetic NOD mice treated with either 0.2 U insulin (Levemir, Novo Nordisk) twice daily, 0.02 U insulin twice daily with and without twice daily leptin injections at decreasing doses from 4.8 to 0.3 mg per day for 28 days. With insulin monotherapy at the 0.2 U dose, HbAlc was normal at 3.9%, but glucose levels varied widely. The SEM of the mean of 100 glucose levels measured during 30 days, an index of glycemic lability, was 100 mg/dl. With the low insulin dose plus leptin, HbAlc was 3.2% and plasma glucose over the 29 days averaged 136 mg/dl with an SEM of 32 mg/dl, indicating far less glucose variability than with insulin alone. Liver TG content on this bihormonal regime measured 34 mg/dl, compared to 314 mg/dl on the low 0.02 U dose without leptin and 51 mg/dl on the high 0.2 U insulin dose.

### Example 3

### Clinical Trial Protocol

Inclusion criteria. Type I Diabetes Mellitus (T1DM) patients (n=6) will be sought with an uncomplicated clinical record for at least 1 year. Diagnosis of type I diabetes will be based on clinical criteria including:
1. Age of onset of diabetes (16 years or younger) with insulin-dependence within 6 months of the onset of diabetes or history of prior episode of ketoacidosis, or previous documentation of positive serum islet cell autoantibodies
2. Age 21-40 years
3. Gender, male and female
4. HbAlc>7%
5. Plasma leptin levels less than the 20^{th} percentile of normal levels in the U.S. population (2.5 ng/ml in males and 7 ng/ml in females)
   Exclusion criteria are as follows:

1. Any known clinical disorder other than uncomplicated T1DM

2. Obesity or overweight

2. Hypoglycemia unawareness

3. Use of prescription drugs other than insulin

4. Current substance abuse

5. Subjects who have a known hypersensitivity to E. coli derived proteins

6. Pregnant or lactating women

7. History of weight loss (>10%) in the last 3 months

Study Design. The study will be conducted as an open-label observational study to assess the efficacy of leptin in T1DM. Patients will be recruited from co-investigator's clinic population which consists largely of patients with T1DM, the UT Southwestern endocrinology fellow's diabetes clinic, where GC is attending physician, and from other academic diabetologists at UT Southwestern Medical Center. Following a screening evaluation to identify eligible patients, they will be followed for a 4-week pre-baseline period without changing their insulin regime in order to establish a baseline state. Patients will be given a standard glucose meter and daily fasting, preprandial and 2 hour post-prandial self-monitored blood glucose (SMBG) values will be obtained throughout the study. Glucose meter data will be downloaded at specified intervals to calculate mean and standard deviation as a measure of glucose exposure and variability, respectively. Patients will also be outfitted with continuous glucose monitors for two weeks before and two weeks after initiation of leptin therapy so that average glycemia and glucose variability can be compared to SMBG data and to maximize patient safety at the initiation of leptin therapy.

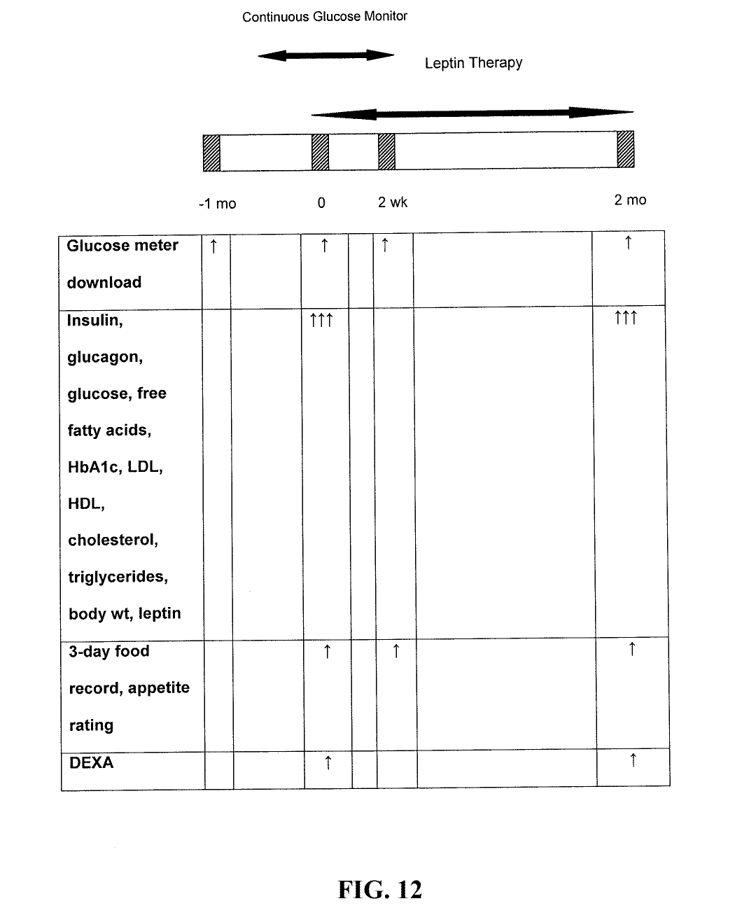

Eligible patients will be treated with their usual regime of diet and human recombinant insulin for 1 month. After a complete stable baseline has been obtained, rmetHuleptin (Amylin Inc.) will be administered at a dose of 0.16 mg/kg body weight/day (in two divided doses) in the female subjects and at a dose of 0.08 mg/kg body weight/day (in two divided doses) in the male subjects. In previous studies of CGL patients, this dose resulted in twice the normal physiological plasma levels of leptin in both females and in males (data not shown). If glucose levels decline (<80 mg/dL), insulin will be adjusted downward in decrements of 5-10% daily, or more if necessary, based on physician review of blood glucose logs. The goal will also be maintain good glycemic control with minimal dose of insulin. If insulin requirements remain the same and do not go down by the end of the second week, the dose of rmetHuleptin will be doubled, and the same evaluation carried out. Patients will receive leptin by subcutaneous injections. They will be observed for 8 hours after the first leptin dose to ensure that leptin does not induce hypoglycemia. Continuous glucose monitoring will be required until stabilization of glucose, insulin and leptin doses have been reached. Initially, the high leptin dose will be employed to achieve the stable euglycemia, at which point the leptin dose will be reduced. The minimal level of hyperleptinemia that is effective in mice will be matched in humans in the hope of similarly normalizing their diabetes. A summary of the treatment and monitoring protocol is provided in FIG. 12.

The following measures will be obtained during the baseline period and at the end of the leptin study period:
1. Glucose monitoring
2. Pharmacodynamic/kinetic leptin profile: After a single leptin injection of 0.08 to 0.16 mg/kg body weight, plasma leptin, glucose, FFA, insulin (free) and glucagon will be measured at 30-60 minute intervals for ˜8-12 h to determine what level of leptin/insulin is required to reverse the abnormal metabolic parameters.
3. 72-hour diet record x3
4. Initial plasma leptin level profile (q 1 h after a leptin injection until the next injection) and fasting leptin levels t.i.w.

Study Medication—Recombinant Human Leptin (A-100). Leptin will be made available by the Amylin Pharmaceuticals, La Jolla, Calif. (see letter of support). An IND (66096; dated 10-21-2002) has been filed by the PI related to Leptin trials.

Compliance with Leptin. Compliance with leptin will be monitored by measuring serum leptin levels during the study.

Primary and Secondary Endpoint variables: The primary endpoint variables will be HbAlc, and mean and standard deviation of blood glucose from glucose meter download. The secondary endpoint variable will be change in total daily insulin dose. The inventor will also assess effects of leptin therapy on energy intake as assessed by 3-day food record and body weight and fat by DEXA. A satiety analysis will be employed. Serum leptin concentrations will be measured to assess adequacy of leptin replacement.

Potential Untoward Effects. The most frequently reported adverse event in studies of r-metHu Leptin has been a skin reaction at the site of injection. The reactions include bruising, redness, pain, itching, inflammation, swelling, dark spots on skin, and lumps under the skin. Other frequently reported adverse events have been headache, fatigue, nausea and influenza-like symptoms. There may be additional risks such as allergic reactions. There have been less frequent reports of generalized rashes, urticaria, and, rarely, angioneurotic edema of the lips and eyes. Occasional patients have developed elevations of hepatic enzymes but these were reversible. There is also the possibility of developing leptin antibodies.

Justification for the Choice of Study Design. An open-labeled, pilot trial is designed to determine if the remarkable benefits of leptin therapy in rodents with T1DM can be translated to humans. A pre-baseline period of 4 weeks has been included to obtain stable measurements of various metabolic parameters, followed by 2 months of therapy. This should be sufficient to indicate if leptin does in human T1DM what it does in rodent T1DM. If the answer is “yes,” a new protocol will be submitted for rigorous, controlled larger study of longer duration.

Sources of Materials. Research material will consist of questionnaires, blood specimens, physical exams, 3 day food recalls, and information obtained from the subjects according to the protocol. No use will be made of existing specimens, records or data.

Potential Risks. All subjects will undergo a history, physical examination, and blood drawing for which there is the minimal risk of psychological or physical discomfort, the inconvenience of time spent and the unlikely risk for bruising, fainting or infection. The potential risks and discomforts of the evaluation and interventional treatment are as follows. DEXA scans: exposure to a small amount of radiation and the need to hold still for about 15 minutes. The 3 day food recall has no associated risk. Leptin therapy: the most common risk is injection site reaction, there have been mild respiratory infections, rare liver enzyme elevations, rare proteinuria, and the development of antibodies to leptin. There is the potential for increased frequency of hypoglycemia, although this has not occurred in patients with CGL and diabetes who were treated with both insulin and leptin.

Protection Against Risk. Potential risks will be minimized by utilizing study codes to identify subjects, all files will be kept locked and all information on computers will be password protected. Access to research data is restricted to key personnel directly involved with the study who have been trained in the protection of human subjects and signed statements assuring their compliance with University policies protecting the privacy of research subjects. The Certificate of Confidentiality will provide additional protection for the research subjects. The physical risks will be minimized by the inclusion and exclusion criteria and a complete physical examination prior to initiating study procedures with careful monitoring throughout. Pregnant women are excluded from studies involving radiation, such as DEXA. For the protection of all subjects, blood tests including chemistry, lipids and liver enzymes will be obtained periodically.

To protect against hypoglycemia, patients with hypoglycemia unawareness will be excluded. Additionally, patients will be outfitted with continuous glucose monitoring (CGM) devices starting 2 weeks prior to the initiation of leptin therapy. This will allow time for the subjects to familiarize themselves with the technology. Alarms on the CGM device will be set so that patients are alerted to blood glucose values as soon as it falls to below 80 mg/dl. Patients will also be instructed to test blood glucose before each meal and 2 hours after insulin bolus. Rapid acting insulin analogs peak in activity 2 hours after injection and so by testing blood glucose at this time, patients can be taught to predict and prevent impending hypoglycemia. Patients will be observed for 8 hours after their first dose of leptin. This will allow enough time to assess response to 2 separate prandial doses of insulin so that if leptin rapidly sensitizes to the effect of insulin, patients will be in an observed setting and insulin doses can immediately be adjusted downward. Blood glucose logs will be reviewed daily for the first week after initiation of leptin and then every 2 weeks until study end. Insulin doses, including basal insulin, insulin to carbohydrate ratio, and insulin to correct for hyperglycemia will be adjusted based on review of blood glucose logs.

Inclusion of women, minorities and children. Women make up 50% of those impacted by type I diabetes. The inventor anticipates enrolling 50% women in our studies. All minorities and ethnicities will be enrolled. Patients for this pilot study will be recruited from the adult diabetes clinics and therefore children will not be included.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. REFERENCES

The following references, to the extent that they provide exemplary procedural or other details supplementary to those set forth herein, are specifically incorporated herein by reference.
U.S. Pat. No. 4,179,337
U.S. Pat. No. 4,797,368
U.S. Pat. No. 5,139,941
U.S. Pat. No. 5,521,283
U.S. Pat. No. 5,525,705
U.S. Pat. No. 5,532,336
U.S. Pat. No. 5,552,522
U.S. Pat. No. 5,552,523
U.S. Pat. No. 5,552,524
U.S. Pat. No. 5,554,727
U.S. Pat. No. 5,559,208
U.S. Pat. No. 5,563,243
U.S. Pat. No. 5,563,243
U.S. Pat. No. 5,563,244
U.S. Pat. No. 5,563,245
U.S. Pat. No. 5,567,678
U.S. Pat. No. 5,567,803
U.S. Pat. No. 5,569,743
U.S. Pat. No. 5,569,744
U.S. Pat. No. 5,574,133
U.S. Pat. No. 5,580,954
U.S. Pat. No. 5,594,101
U.S. Pat. No. 5,594,104
U.S. Pat. No. 5,605,886
U.S. Pat. No. 5,614,379
U.S. Pat. No. 5,691,309
U.S. Pat. No. 5,719,266
U.S. Pat. No. 5,831,017
U.S. Pat. No. 5,840,517
U.S. Pat. No. 5,851,995
U.S. Pat. No. 5,856,098
U.S. Pat. No. 5,919,902
U.S. Pat. No. 5,935,810
U.S. Pat. No. 5,972,888
U.S. Pat. No. 6,001,968
U.S. Pat. No. 6,007,998
U.S. Pat. No. 6,221,838
U.S. Pat. No. 6,350,730
U.S. Pat. No. 6,395,509
U.S. Pat. No. 6,420,339
U.S. Pat. No. 6,429,290
U.S. Pat. No. 6,541,033
U.S. Pat. No. 6,777,388
U.S. Pat. No. 6,936,439
U.S. Pat. No. 7,112,659
U.S. Pat. No. 7,186,694
U.S. Pat. No. 7,208,572
U.S. Patent Publn. 2003/0092126
U.S. Patent Publn. 2003/0166847
U.S. Patent Publn. 2003/049693
U.S. Patent Publn. 2004/0072219
U.S. Patent Publn. 2005/0163799
U.S. Patent Publn. 2005/0176107
Abuchowski et al., in Enzymes as Drugs. (J. S. Holcerberg and J. Roberts, eds. pp. 367-383, 1981.
Baichwal et al., In: Gene transfer, Kucherlapati (Ed), Nyork, Plenum Press, 117-148, 1986.
Banting and Best, J. Lab. Clin. Med., 115(2):254-272, 1990.
Bauer et al., Blood 86:2379-2387, 1995.
Becker et al., Methods Cell Biol., 43 Pt A:161-89, 1994b.
Becker et al., J. Biol. Chem., 269(33):21234-8, 1994a.
Becker et al., J. Biol. Chem., 271:390-394, 1996.
Bestwick et al., Proc. Natl. Acad. Sci. USA, 85:5404-5408, 1988.
Briggs et al., Health Econ., 12(5):377-392, 1993.
Chang et al., Hepatology, 14:134 A, 1991.
Chen et al., Proc. Natl. Acad. Sci. USA, 101(31):11245-11250, 2004,
Chinookoswong et al., Diabetes, 48(7):1487-1492, 1999.
Choi et al., Glia, 56(7):791-800, 2008.
Christ et al., Eur. J. Biochem., 178(2):373-379, 1989.
Clark et al., Human Gene Therapy, 6:1329-1341, 1995.
Coffin, In: Virology, Fields et al. (Eds.), NY, Raven Press, 1437-1500, 1990.
Cohen et al., Protein Science 4:1088, 1995.
Cotten et al., Proc. Natl. Acad. Sci. USA, 89:6094-6098, 1992.
Couch et al., Am. Rev. Resp. Dis., 88:394-403, 1963.
Coupar et al., Gene, 68:1-10, 1988.
Curiel, In: Viruses in Human Gene Therapy, Vos (Ed.), Carolina Academic Press, Durham, N.C., 179-212, 1994.
Dalle et al., J. Biol. Chem., 279(19):20345-20355, 2004.
Dobbs et al., Science, 187(4176):544-547, 1975.
EP 0273085
EP 0401384
Exton et al., J. Biol. Chem., 244(15):4095-4102, 1969.
Ferkol et al., FASEB J., 7:1081-1091, 1993.
Flotte et al., Am. J. Respir. Cell Mol. Biol., 7:349-356, 1992.
Flotte et al., Gene Therapy, 2:29-37, 1995.
Flotte et al., Proc. Natl. Acad. Sci. USA, 90:10613-10617, 1993.
Flowers et al., Proc. Natl. Acad. Sci. USA, 87:2349-2353, 1990.
Folch et al., J. Biol. Chem., 226(1):497-509, 1957.
Fraley et al., Proc. Natl. Acad. Sci. USA, 76:3348-3352, 1979.
Friedmann, Science, 244:1275-1281, 1989.
Gainer et al., Transplantation, 61(11):1567-1571, 1996.
Gallichan et al., Hum. Gene Ther., 9:2717-2726, 1998.
Gerich et al., N. Engl. J. Med., 292(19):985-989, 1975.
Ghosh et al., In: Liver diseases, targeted diagnosis and therapy using specific receptors and ligands, Wu and Wu (Ed.), NY, Marcel Dekker, 87-104, 1991.
Ghosh-Choudhury et al., EMBO J., 6:1733-1739, 1987.
Gomez-Foix et al., J. Biol. Chem., 267:25129-25134, 1992.
Graham et al., Biotechnology, 20:363-390, 1992.
Graham et al., In: Methods in Molecular Biology: Gene Transfer and Expression Protocol, Murray (Ed.), Clifton, N.J., Humana Press, 7:109-128, 1991.
Graham et al., J. Gen. Virol., 36:59-72, 1977.
Grunhaus et al., Seminar in Virology, 3:237-252, 1992.
Hardie et al., Annu. Rev. Biochem., 67:821-855, 1998.
Hermonat and Muzyczka, Proc. Natl. Acad. Sci. USA, 81:6466-6470, 1984.
Hersdorffer et al., DNA Cell Biol., 9:713-723, 1990.
Herz and Gerard, Proc. Natl. Acad. Sci. USA, 90:2812-2816, 1993.
Hidaka et al., FASEB J., 16(6):509-518, 2002.
Higa et al., Proc. Natl. Acad. Sci. USA, 96(20):11513-11518, 1999.
Horwich et al. J. Virol., 64:642-650, 1990.
Jones and Shenk, Cell, 13:181-188, 1978.
Kafri et al., J. Virol. 73:570-584, 1999.
Kaneda et al., Science, 243:375-378, 1989.
Kaplitt et al., Nature Genetics, 8:148-154, 1994.
Karlsson et al., EMBO J., 5:2377-2385, 1986.
Kato et al., J. Biol. Chem., 266:3361-3364, 1991.
Kelleher and Vos, Biotechniques, 17(6):1110-1117, 1994.
Kharitonenkov et al., J. Cell Physiol., 215(1):1-7, 2008.
Kiem et al., Blood 83:1467-1473, 1994.
Kim et al., Nat. Med., 10(7):727-733, 2004.
Klein et al., Nature, 327:70-73, 1987.
Kojima et al., Proc. Natl. Acad. Sci. USA, 101(8):2458-2463, 2004.
Kotin et al., Proc. Natl. Acad. Sci. USA, 87:2211-2215, 1990.
Koyama et al., Diabetes, 46(8):1276-1280, 1997.
LaFace et al., Virology, 162:483-486, 1988.
Laughlin et al., J. Virol., 60:515-524, 1986.
Le Gal La Salle et al., Science, 259:988-990, 1993.
Lebkowski et al., Mol. Cell. Biol., 8:3988-3996, 1988.
Lee et al., Proc. Natl. Acad. Sci. USA, 99(18):11848-11853, 2002.
Levrero et al., Gene, 101:195-202, 1991.
Lin et al., Am. J. Physiol. Endocrinol. Metab., 282(5):E1084-1091, 2002.
Luo et al., Blood, 82(Supp.):1,303A, 1994.
Malik et al., Exp. Hematol. 20:1028-1035, 1992.
Mann et al., Cell, 33:153-159, 1983.
Markowitz et al., J. Virol., 62:1120-1124, 1988.
Matsumoto et al., Cell Metab., 6(3):208-216, 2007.
McCarty et al., J. Virol., 65:2936-2945, 1991.
McLaughlin et al., J. Virol., 62:1963-1973, 1988.
Melloul et al., Proc. Natl. Acad. Sci. USA, 90(9):3865-9, 1993.
Miller and Rosman, BioTechniques, 7:980-988, 1989.
Miller et al., Meth. Enzymol., 217:581-599, 1993.
Minokoshi et al., Nature, 415(6869):339-343, 2002.
Miyanaga et al., Diabetologia, 46:1329-1337, 2003.
Miyoshi et al., J. Virol., 72:8150-8157, 1998.
Muller et al., J. Clin. Invest., 50(9):1992-1999, 1971.
Murakami et al., Biochem. Biophys. Res. Comm. 209: 944-952, 1995.
Muzyczka, Curr. Top. Microbiol. Immunol., 158:97-129, 1992.
Nicolas and Rubinstein, In: Vectors: A survey of molecular cloning vectors and their uses, Rodriguez and Denhardt (Eds.), Stoneham, Butterworth, 494-513, 1988.
Nicolau and Sene, Biochim. Biophys. Acta, 721:185-190, 1982.
Nicolau et al., Methods Enzymol., 149:157-176, 1987.
Oge et al., Diabetologia, 50(5):1099-1108, 2007.
Ohi et al., Gene, 89L:279-282, 1990.
Oldstone et al., J. Exp. Med., 171(6):2091-2100, 1990.
Oral et al., N. Engl. J. Med., 346(8):570-578, 2002.
Orchard et al., Diabetes Care, 26(5):1374-1379, 2003.
Orci et al., Proc. Natl. Acad. Sci. USA, 101(7):2058-2063, 2004.
Orci et al., Proc. Natl. Acad. Sci. USA, 73(4):1338-1342, 1976.
Osborne et al., Hum. Gene Ther., 1:31-41, 1990.
Paskind et al., Virology, 67:242-248, 1975.
PCT Appln. WO 04/039832
PCT Appln. WO 00/09165
PCT Appln. WO 00/20872
PCT Appln. WO 00/21574
PCT Appln. WO 00/47741
PCT Appln. WO 96/05309
PCT Appln. WO 96/40912
PCT Appln. WO 97/06816
PCT Appln. WO 97/18833
PCT Appln. WO 97/38014
PCT Appln. WO 98/08512
PCT Appln. WO 98/28427
PCT Appln. WO 98/46257
PCT Appln. WO 96/05309
Perales et al., Proc. Natl. Acad. Sci. USA, 91:4086-4090, 1994.
Racher et al., Biotechnology Techniques, 9:169-174, 1995.
Ragot et al., Nature, 361:647-650, 1993.
Renan, Radiother. Oncol., 19:197-218, 1990.
Rich et al., Hum. Gene Ther., 4:461-476, 1993.
Ridgeway, In: A survey of molecular cloning vectors and their uses, Rodriguez et al. (Ed.), Stoneham, Butterworth, 467-492, 1988.
Rosenfeld et al., Cell, 68:143-155, 1992.
Rosenfeld et al., Science, 252:431-434, 1991.
Roux et al., Proc. Natl. Acad. Sci. USA, 86:9079-9083, 1989.
Sambrook et al., “Molecular Cloning: A Laboratory Manual,” Nolan, (Ed.), Cold Spring Harbor Laboratory Press, Cold Spring Harbor, N.Y., 1989.
Samulski et al., EMBO J., 10:3941-3950, 1991.
Samulski et al., J. Virol., 63:3822-3828, 1989.
Sapir et al., Proc. Natl. Acad. Sci. USA, 102(22):7964-7969, 2005.
Shelling and Smith, Gene Therapy, 1:165-169, 1994.
Shimomura et al., Mol. Cell, 6(1):77-86, 2000.
Stockschlaeder et al., Hum. Gene Ther. 2:33-39, 1991.
Stratford-Perricaudet and Perricaudet, In: Human Gene Transfer, Cohen-Haguenauer and Boiron (Eds.), John Libbey Eurotext, France, 51-61, 1991.
Stratford-Perricaudet et al., Hum. Gene Ther., 1:241-256, 1990.
Szanto and Kahn, Proc. Natl. Acad. Sci. USA, 97(5):2355-2360, 2000.
Temin, In: Gene Transfer, Kucherlapati (Ed.), NY, Plenum Press, 149-188, 1986.
Top et al., J. Infect. Dis., 124:155-160, 1971.
Tratschin et al., Mol. Cell. Biol., 4:2072-2081, 1984.
Tratschin et al., Mol. Cell. Biol., 5:32581-3260, 1985.
Tuduri et al., Diabetes, 58(7):1616-1624, 2009.
Unger and Orci, Lancet., 1(7897):14-16, 1975.
Wagner et al., Science, 260:1510-1513, 1990.
Walsh et al., J. Clin. Invest., 94:1440-1448, 1994.
Wang et al., Zhonghua Shi Yan He Lin Chuang Bing Du Xue Za Zhi., 22(6):504-506, 2008.
Wei et al., Gene Therapy, 1:261-268, 1994.
Wong et al., Gene, 10:87-94, 1980.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Yang et al., J. Virol., 68:4847-4856, 1994a.
Yang et al., Proc. Natl. Acad. Sci. USA, 87:9568-9572, 1990.
Yoder et al., Blood, 82(Supp.):1:347 A, 1994.
Zhang et al., Nature, 372(6505):425-432, 1990.
Zhou et al., Exp. Hematol., 21:928-933, 1993.
Zhou et al., J. Exp. Med., 179:1867-1875, 1994.

1. A method of treating type I diabetes comprising providing to a subject diagnosed with type I diabetes a therapeutically effective amount of (a) leptin, a leptin agonist, or a leptin derivative; and (b) no more than about 10% of a normal daily dosage of insulin supplementation.

2. The method of claim 1, wherein said subject is a human.

3. The method of claim 1, wherein said subject is a non-human animal.

4. The method of claim 1 or claim 2, wherein said non-human animal is a mouse or rat.

5. The method of any one of claims 1 through 4, wherein said subject suffers from autoimmune type I diabetes.

6. The method of any one of claims 1 through 4, wherein said subject suffers from chemically-induced type I diabetes.

7. The method of any one of claims 1 through 5, wherein providing comprises administering leptin, a leptin agonist, or a leptin derivative to said subject.

8. The method of any one of claims 1 through 7, wherein said leptin, leptin agonist, or leptin derivative comprises a polypeptide that has 83 percent or greater amino acid sequence identity to the amino acid sequence set out SEQ ID NO: 13, 14, or 15.

9. The method of any one of claims 1 through 8, wherein said leptin agonist comprises metreleptin (SEQ ID NO: 13).

10. The method of any one of claims 1 through 9, wherein providing comprises administering an expression cassette comprising a promoter operably linked to a leptin-encoding nucleic acid or a leptin agonist-encoding nucleic acid to said subject.

11. The method of claim 10, wherein said promoter is a tissue specific or constitutive promoter.

12. The method of claim 10, wherein said expression cassette is comprised within a lipid vehicle.

13. The method of claim 10, wherein said expression cassette is comprised within a replicable expression construct.

14. The method of claim 13, wherein said replicable expression construct is a non-viral construct.

15. The method of claim 13, wherein said replicable expression construct is a viral construct.

16. The method of claim 15, wherein said viral construct is an adenoviral construct, an adeno-associated viral construct, a pox-viral construct, a retroviral construct, or a herpesviral construct.

17. The method any one of claims 1 through 16, wherein said one or more of the following diabetic symptoms are improved: excess gluconeogenesis, excess glycogenolysis, hyperglycemia, hyperglucagonemia, ketosis, diabetic ketoacidosis, hypertriglyceridemia, elevated plasma free fatty acid, weight loss, catabolic syndrome, terminal illness, hypertension, diabetic nephropathy, renal insufficiency, renal failure, hyperphagia, muscle wasting, diabetic neuropathy, diabetic retinopathy, or diabetic coma.

18. The method of any one of claims 1 through 17, wherein the insulin daily dosage is 10-15%, inclusive, of the normal daily dosage.

19. The method of any one of claims 1 through 18, wherein the insulin daily dosage is 5-10%, inclusive, of the normal daily dosage, inclusive.

20. The method any one of claims 1 through 19, wherein the insulin dosage is less than 5% of the normal daily dosage.

21. The method any one of claims 1 through 20, wherein the insulin dosage is between 0% and 5%, inclusive, of the normal daily dosage.

22. The method of any one of claims 1 through 21, wherein no exogenous insulin is provided.

23. The method of any one of claims 1 through 22, wherein said subject is essentially devoid of endogenous insulin.

24. The method of any one of claims 1 through 23, wherein said subject is has uncontrolled type I diabetes.

25. The method of any one of claims 1 through 21, 23, and 24, wherein the insulin dosage is reduced following initiation of leptin or leptin agonist provision.

26. The method of any one of claims 1 through 25, wherein providing comprises systemically administering to said subject said leptin, said leptin agonist, or said expression cassette.

27. The method of any one of claims 1 through 26, wherein systemically administering comprises intravenous, intra-muscular, subcutaneous, intraperitoneal, transdermal or intra-arterial administration.

28. The method of any one of claims 1 through 27, wherein providing comprises administering directly to a tissue of said subject leptin, said leptin agonist or said expression cassette.

29. The method of claim 28, wherein said tissue is muscle or liver.

30. The method of any one of claims 1 through 29, wherein providing comprises multiple administrations of said leptin, said leptin agonist or said expression cassette.

31. The method of claim 30, wherein multiple administrations comprise 2, 3, 4, 5, 6, 7, 8, 9, 10, 15, 20, 25, 30, 40, 50, 75, 100, 200, 300, 400, 500 or more administrations.

32. The method of claim 30 or claim 31, wherein multiple administrations are separated by 6 hours, 12 hours, 1 day, 2 days, 3, days, 4, days, 5 days, 6 days, 1 week, 2 weeks, one month, two months, three months, 6 months or more.

33. The method of any one of claims 1 through 32, wherein said providing achieves plasma leptin levels of greater than 10 ng/ml, 20 ng/ml, 50 ng/ml, 100 ng/ml, 200 ng/ml, 300 ng/ml or 400 ng/ml.

34. The method of any one of claims 1 through 33, wherein said providing achieves a venous or capillary fasting blood glucose (FBG) levels of less than 200 mg/dl, less than 175 mg/dl, less than 150 mg/dl, less than 140 mg/dl, less than 130 mg/dl, less than 126 mg/dl, less than 120 mg/dl, or less than 115 mg/dl, less than 110 mg/dl, or less than 100 mg/dl.

35. A method of restoring normoglycemia in a subject diagnosed with type I diabetes comprising inducing hyperleptinemia in said subject, wherein said inducing comprises the provision of a therapeutically effective amount of a leptin, a leptin agonist, or a leptin derivative, wherein said subject receives no more than about 10% of a normal daily dosage of insulin supplementation.

36. The method of any one of claims 1 through 35, wherein said hyperleptinemia is characterized by plasma leptin levels of greater than 10 ng/ml, 20 ng/ml, 50 ng/ml, 100 ng/ml, 200 ng/ml, 300 ng/ml or 400 ng/ml.

37. The method of any one of claims 1 through 35, wherein said method results in a venous or capillary fasting blood glucose (FBG) levels of less than 200 mg/dl, less than 175 mg/dl, less than 150 mg/dl, less than 140 mg/dl, less than 130 mg/dl, less than 126 mg/dl, less than 120 mg/dl, or less than 115 mg/dl, less than 110 mg/dl, or less than 100 mg/dl.

38. A method of reducing, suppressing, attenuating, or inhibiting hyperglucogonemia or a condition associated with hyperglucogonemia in a subject diagnosed with type I diabetes comprising inducing hyperleptinemia in said subject, said inducing comprising providing a therapeutically effective amount of a leptin protein, a leptin agonist, or a leptin derivative, wherein said subject receives no more than about 10% of a normal daily dosage of insulin supplementation.

39. The method of any of claim 38, wherein said hyperleptinemia is characterized by plasma leptin levels of greater than 10 ng/ml, 20 ng/ml, 50 ng/ml, 100 ng/ml, 200 ng/ml, 300 ng/ml or 400 ng/ml.

40. The method of claim 38 or 39, wherein said method results in a venous or capillary fasting blood glucose (FBG) levels of less than 200 mg/dl, less than 175 mg/dl, less than 150 mg/dl, less than 140 mg/dl, less than 130 mg/dl, less than 126 mg/dl, less than 120 mg/dl, or less than 115 mg/dl, less than 110 mg/dl, or less than 100 mg/dl.

41. A method of reducing HbAlc in a subject having type I diabetes comprising inducing hyperleptinemia in said subject, said inducing comprising providing a therapeutically effective amount of a leptin protein, a leptin agonist, or a leptin derivative, wherein said subject receives no more than about 10% of a normal daily dosage of insulin supplementation.

42. The method of any of claim 41, wherein said hyperleptinemia is characterized by plasma leptin levels of greater than 10 ng/ml, 20 ng/ml, 50 ng/ml, 100 ng/ml, 200 ng/ml, 300 ng/ml or 400 ng/ml.

43. The method of claim 41 or 42, wherein said method results in a venous or capillary fasting blood glucose (FBG) levels of less than 200 mg/dl, less than 175 mg/dl, less than 150 mg/dl, less than 140 mg/dl, less than 130 mg/dl, less than 126 mg/dl, less than 120 mg/dl, or less than 115 mg/dl, less than 110 mg/dl, or less than 100 mg/dl.

44. The method of any one of claims 1 through 43, wherein said therapeutically effective amount of said leptin protein, leptin agonist, or leptin derivative comprises: about 1 μg per day; about 5 μg per day; about 10 μg per day; about 50 μg per day to about 100 μg per day; about 500 μg per day; about 1 mg per day; about 5 mg per day; about 10 mg per day; about 50 mg per day; or about 100 mg per day.

45. The method of any one of claims 1 through 44, wherein said therapeutically effective amount of said leptin protein, leptin agonist, or leptin derivative comprises a daily dosage of from about 0.01 mg/kg to about 100 mg/kg; from about 0.01 mg/kg to about 80 mg/kg; from about 0.01 mg/kg to about 70 mg/kg; from about 0.01 mg/kg to about 60 mg/kg; from about 0.01 mg/kg to about 50 mg/kg; from about 0.01 mg/kg to about 40 mg/kg; from about 0.01 mg/kg to about 30 mg/kg; from about 0.01 mg/kg to about 25 mg/kg; from about 0.01 mg/kg to about 20 mg/kg; from about 0.01 mg/kg to about 15 mg/kg; from about 0.01 mg/kg to about 10 mg/kg; from about 0.01 mg/kg to about 5 mg/kg; from about 0.01 mg/kg to about 3 mg/kg; from about 0.01 mg/kg to about 1 mg/kg; from about 0.01 mg/kg to about 0.3 mg/kg from about 100 mg/kg to about 90 mg/kg; from about 100 mg/kg to about 80 mg/kg; from about 100 mg/kg to about 70 mg/kg; from about 100 mg/kg to about 60 mg/kg; from about 100 mg/kg to about 50 mg/kg; from about 100 mg/kg to about 40 mg/kg; from about 85 mg/kg to about 10 mg/kg; from about 75 mg/kg to about 20 mg/kg; from about 65 mg/kg to about 30 mg/kg; from about 55 mg/kg to about 35 mg/kg; from about 55 mg/kg to about 45 mg/kg; or from about 0.01 mg/kg to about 20 mg/kg.

46. The method of any one of claims 1 through 45, wherein said therapeutically effective amount is provided by injection of a single dose or in divided doses.

47. The method of any one of claims 1 through 45, wherein said therapeutically effective amount is provided by continuous infusion at an infusion rate of: from about 0.01/pmol/kg/min to about 10 pmol/kg/min.

50. A method of treating type1 diabetes comprising providing to a subject diagnosed with type I diabetes a therapeutically effective amount of (a) leptin, a leptin agonist, or a leptin derivative; wherein said subject is essentially devoid of endogenous insulin.

51. A method of treating type I diabetes comprising providing to a subject diagnosed with type I diabetes a therapeutically effective amount of (a) leptin, a leptin agonist, or a leptin derivative; in the absence of exogenous insulin.

52. The method of claim 51 or claim 52, wherein said subject is a human.

53. The method of claim 51 or claim 52, wherein said subject is a non-human animal.

54. The method of any one of claim 51 through 53, wherein said non-human animal is a mouse or rat.

55. The method of any one of claims 51 through 54, wherein said subject suffers from autoimmune type I diabetes.

56. The method of any one of claims 51 through 54, wherein said subject suffers from chemically-induced type I diabetes.

57. The method of any one of claims 51 through 56, wherein providing comprises administering leptin, a leptin agonist, or a leptin derivative to said subject.

58. The method of any one of claims 51 through 57, wherein said leptin, leptin agonist, or leptin derivative comprises a polypeptide that has 83 percent or greater amino acid sequence identity to the amino acid sequence set out SEQ ID NO: 13, 14, or 15.

59. The method of any one of claims 51 through 88, wherein said leptin agonist comprises metreleptin (SEQ ID NO: 13).

60. A method of reducing, suppressing, attenuating, or inhibiting hyperglucogonemia or a condition associated with hyperglucogonemia in a subject diagnosed with type I diabetes comprising inducing hyperleptinemia in said subject, said inducing comprising providing a therapeutically effective amount of a leptin protein, a leptin agonist, or a leptin derivative, wherein said subject is essentially devoid of endogenous insulin.

61. A method of reducing, suppressing, attenuating, or inhibiting hyperglucogonemia or a condition associated with hyperglucogonemia in a subject diagnosed with type I diabetes comprising inducing hyperleptinemia in said subject, said inducing comprising providing a therapeutically effective amount of a leptin protein, a leptin agonist, or a leptin derivative, in the absence of exogenous insulin.

62. The method of any one of claim 60 or claim 61, wherein said hyperleptinemia is characterized by plasma leptin levels of greater than 10 ng/ml, 20 ng/ml, 50 ng/ml, 100 ng/ml, 200 ng/ml, 300 ng/ml or 400 ng/ml.

63. The method of any one of claims 60 through 62, wherein said hyperglucogonemia or a condition associated with hyperglucogonemia comprises at least one of the following: excess gluconeogenesis, excess glycogenolysis, hyperglycemia, hyperglucagonemia, ketosis, diabetic ketoacidosis, hypertriglyceridemia, elevated plasma free fatty acid, weight loss, catabolic syndrome, terminal illness, hypertension, diabetic nephropathy, renal insufficiency, renal failure, hyperphagia, muscle wasting, diabetic neuropathy, diabetic retinopathy, or diabetic coma

64. The method of any one of claims 60 through 63, wherein said subject is a human.

65. The method of any one of claims 60 through 63, wherein said subject is a non-human animal.

66. The method of claim 65, wherein said non-human animal is a mouse or rat.

67. The method of any one of claims 60 through 66, wherein said subject suffers from autoimmune type I diabetes.

68. The method of any one of claims 60 through 66, wherein said subject suffers from chemically-induced type I diabetes.

69. The method of any one of claims 60 through 68, wherein providing comprises administering leptin, a leptin agonist, or a leptin derivative to said subject.

70. The method of any one of claims 60 through 69, wherein said leptin, leptin agonist, or leptin derivative comprises a polypeptide that has 83 percent or greater amino acid sequence identity to the amino acid sequence set out SEQ ID NO: 13, 14, or 15.

71. The method of any one of claims 60 through 70, wherein said leptin agonist comprises metreleptin (SEQ ID NO: 13).


SEQUENCE LISTING (15 sequences)

SEQ ID NO: 1 - DNA, 17 bp, Artificial
  Synthetic primer
ggccactggccgctatt

SEQ ID NO: 2 - DNA, 21 bp, Artificial
  Synthetic primer
gcgacttcatcctttgcaatg

SEQ ID NO: 3 - DNA, 21 bp, Artificial
  Synthetic primer
attcatttcgcgtttggaaaa

SEQ ID NO: 4 - DNA, 20 bp, Artificial
  Synthetic primer
cagacccagcacggaaagaa

SEQ ID NO: 5 - DNA, 20 bp, Artificial
  Synthetic primer
gcctgtgggaaaaccaacct

SEQ ID NO: 6 - DNA, 22 bp, Artificial
  Synthetic primer
cacccacacattcaactttcca

SEQ ID NO: 7 - DNA, 21 bp, Artificial
  Synthetic primer
cagccagtacagccctgatga

SEQ ID NO: 8 - DNA, 19 bp, Artificial
  Synthetic primer
tggtaagcgcagccaagag

SEQ ID NO: 9 - DNA, 22 bp, Artificial
  Synthetic primer
tttgtcaaacagcacctttgtg

SEQ ID NO: 10 - DNA, 23 bp, Artificial
  Synthetic primer
gggtgtgtagaagaaaccacgtt

SEQ ID NO: 11 - DNA, 20 bp, Artificial
  Synthetic primer
ggggagcgtggcttcttcta

SEQ ID NO: 12 - DNA, 20 bp, Artificial
  Synthetic primer
ggggacagaattcagtggca

SEQ ID NO: 13 - protein, 147 aa, Homo sapiens
MVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 14 - protein, 146 aa, Homo sapiens
MVPIQKVQDDTKTLIKTIVTRINDISHTSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 15 - protein, 147 aa, Homo sapiens
MVPIQKVQDDTKTLIKTIVTRINDISHTQSVSAKQRVTGLDFIPGLHPILSLSKMDQTLAVYQQVLTSLPSQNVLQIANDLENLRDLLHLLAFSKSCSLPQTSGLQKPESLDGVLEASLYSTEVVALSRLQGSLQDILQQLDVSPEC